(19) United States
(12) Patent Application Publication
(10) Pub. No.: US 2020/0253916 A1
(43) Pub. Date: Aug. 13, 2020

(54) Compositions of Azadirachta Indica and Methods of Treating Cancer

(71) Applicant: Board of Regents of the University of Texas System, Austin, TX (US)

(72) Inventor: Michael Wargovich, San Antonio, TX (US)

(21) Appl. No.: 16/557,028

(22) Filed: Aug. 30, 2019

Related U.S. Application Data
(60) Provisional application No. 62/725,484, filed on Aug. 31, 2018.

(30) Foreign Application Priority Data
Sep. 6, 2018 (IN) 201821021206

(51) Int. Cl. A61K 31/343; A61K 31/355; A61K 36/185; A61K 35/04

(52) U.S. Cl. CPC A61K 31/343; A61K 31/355; A61K 9/48; A61K 35/04; A61K 36/185

(57) ABSTRACT

The disclosure relates to compositions and methods of treating cancer in a subject. The method comprises administering to a patient in need of treatment an effective amount of supercritical CO₂ neem extract.

### CROSS-REFERENCE TO RELATED APPLICATIONS

This application claims the benefit of the filing date of U.S. Provisional Application No. 62/725,484 which was filed on Aug. 31, 2018, and Indian Application No. 201821021206 which was filed on Sep. 6, 2019. The content of these earlier filed applications are hereby incorporated herein by reference in their entirety.

### BACKGROUND

The risk for developing oral cancers is increasing world-wide as the global rise in tobacco use, alcohol consumption, and HPV exposure continues. Conventional treatments have improved the 5-year survival rates for patients with early disease, while patients with late-stage disease have a 5-year survival rate as low as 34% which has not changed in nearly 40 years.

Colorectal cancer (CRC) is the third most commonly diagnosed cancer and the second leading cause of cancer death in men and women in the United States. Anti-inflammatory blockade has been proven to be a promising avenue of colorectal cancer prevention. However, NSAIDs, while effective in curbing CRC risk, are too toxic for long term use in cancer prevention.

### SUMMARY

Disclosed herein are methods of treating cancer in a subject, the methods comprising: (a) identifying a subject in need of treatment; and (b) administering to the subject a composition comprising a therapeutically effective amount of a supercritical CO₂ neem extract (SCNE), wherein the SCNE comprises nimbolide, nimbin and salinin.

Disclosed herein are methods of reducing at least one inflammatory cytokine in serum of a subject in need thereof, the methods comprising administering to the subject a composition comprising a therapeutically effective amount of a supercritical CO₂ neem extract (SCNE), wherein the SCNE comprises nimbolide, nimbin and salinin.

Disclosed herein are methods of reducing inflammation in a subject in need thereof, the methods comprising administering to the subject a composition comprising a therapeutically effective amount of a supercritical CO₂ neem extract (SCNE), wherein the SCNE comprises nimbolide, nimbin and salinin.

Disclosed herein are methods of treating a hyperproliferative disorder in a subject in need thereof, the methods comprising administering to the subject a composition comprising a therapeutically effective amount of a supercritical CO₂ neem extract (SCNE), wherein the SCNE comprises nimbolide, nimbin and salinin.

Disclosed herein are methods of suppressing expression of NFkB and cycloxygenase in a subject in need thereof, the methods comprising administering to the subject a composition comprising a therapeutically effective amount of a supercritical CO₂ neem extract (SCNE), wherein the SCNE comprises nimbolide, nimbin and salinin.

Disclosed herein are methods of suppressing expression of NFkB and cyclooxygenase in at least one cell, the methods comprising the step of contacting at least one cell with an effective amount of a supercritical CO₂ neem extract (SCNE), wherein the SCNE comprises nimbolide, nimbin and salinin.

Disclosed herein are methods of modifying epidermal growth factor receptor signaling activity in a subject, the methods comprising administering to the subject a composition comprising a therapeutically effective amount of a supercritical CO₂ neem extract (SCNE), wherein the SCNE comprises nimbolide, nimbin and salinin.

Disclosed herein are methods of inducing apoptosis of a cell in a subject in need thereof, the methods comprising administering to the subject a composition comprising a therapeutically effective amount of a supercritical CO₂ neem extract (SCNE), wherein the SCNE comprises nimbolide, nimbin and salinin.

Other features and advantages of the present compositions and methods are illustrated in the description below, the drawings, and the claims.

### BRIEF DESCRIPTION OF THE DRAWINGS

FIGS. 1A-F shows SCNE and Nimbolide inhibit oral squamous cancer cell growth. FIG. 1A shows SCNE treatment (0-400 μg/ml) for 8 h or 24 h on SCC4, HSC3, Cal27 OSCC cell lines. FIG. 1B shows SCNE treatment (0-100 μM) for 8 h or 24 h on SCC4, HSC3, Cal27 OSCC cell lines. FIG. 1C shows Nimbolide treatment (0-400 μg/ml) for 48 h on SCC4, HSC3, Cal27 OSCC cell lines. FIG. 1D shows Nimbolide treatment (0-100 μM) for 48 h on SCC4, HSC3, Cal27 OSCC cell lines. FIG. 1E shows Celecoxib treatment (0-200 μM) for 8 h or 24 h on SCC4, HSC3, Cal27 OSCC cell lines. FIG. 1F shows Celecoxib treatment (0-200 μM) for 48 h on SCC4, HSC3, Cal27 OSCC cell lines.

FIG. 2 shows SCNE and Nimbolide downregulate inflammatory mediators. SCC4, Cal27, and HSC3 cells were treated with 20 μg, 60 μg SCNE, or 10 μM, 50 μM Nimbolide for 24 h. Cytosolic protein fractions were analyzed for COX1, COX2, NFkBp65, STAT3, pSTAT3, EGFR, pEGFR, pERK1/2, AKT, and Pakt. Nuclear protein fractions were analyzed for pERK1/2, STAT3, pSTAT3, and NFkBp65. GapDH and Topo-IIα were used as loading controls.

FIGS. 3A-C shows that SCNE and Nimbolide inhibit in vitro cell migration. FIG. 3A shows using the Scratch assay that 60 μg/ml SCNE and 50 mM Nimbolide inhibits cell migration in SCC4 (120 h), Cal27 (72 h) and HSC3 (8 h). Green lines represent initial scratch front, yellow is scratch front after respective treatment time. FIG. 3B shows that the average wound width in SCC4, Cal27, and HSC3 is significantly reduced (n=6, *p<0.05, **p<0.01, ****p<0.001) with SCNE and Nimbolide treatments. FIG. 3C depicts a gelatinase zymograms showing MMP2 and MMP9 activity from SCC4, Cal27, and HSC3 treated cells.

FIG. 4A-B shows that SCNE and Nimbolide inhibit OSCC derived tumor growth in mice. FIG. 4A shows that SCC4 (30 day treatment—81.12% reduction in tumor volume), HSC3 (25 day treatment—48.81% reduction in tumor volume), and Cal27 (35 day treatment—49.00% reduction in tumor volume) cell growth was significantly inhibited in mice fed SCNE 200 mg/kg diet (*p<0.005, **p<0.001). FIG. 4B shows that Nimbolide treatment (20 mg/kg IP) significantly (*p<0.05) reduced (66%) HSC3 tumor volume in mice after 25 days.

FIGS. 5A-C shows that SCNE inhibits tumor growth in 4NQO-1 mouse model of OSCC. FIG. 6A shows that CBA mice showed no difference in weight gain on 200 mg/kg SCNE diet over the 12 week study. FIG. 5B shows that the SCNE diet reduced tongues 5 fold (**p<0.01) and tongue carcinomas compared to no SCNE diet. FIG. 5C shows that the SCNE diet reduced levels of proliferating markers PCNA, Ki-67, c-Met in mouse tongues.

FIG. 6. Shows the effects on mouse circulating cytokine levels from SCC4, Cal27, and HSC3 xenografts in mice fed SCNE.

FIG. 7 shows the effects on mouse circulating cytokine levels from a 4NQO-1 carcinogen induced model of OSCC.

FIG. 8 shows that SCNE reduces serum level inflammatory cytokines in xenografted and carcinogen induced mouse models of OSCC.

FIGS. 9A-D shows that SCNE reduced viability in CRC cells. HCT116, HT29 and IEC6 cells were treated with different concentrations of SCNE (A, B) and nimbolide (C, D) for 48 and 72 hrs and the cell viability was measured by MTT assay. Data were expressed as the mean±SD. from three independent experiments. *P<0.05 indicates significant difference versus vehicle control.

FIGS. 10A-B shows that SCNE induced apoptosis of HCT116 and HT29 cells. CRC cells were treated with SCNE (40 and 75 μg/ml) and nimbolide (5 and 10 μM) for 48 hrs. Expression of apoptosis regulatory proteins in HCT116 and HT29 cells treated with SCNE (A) and nimbolide (B) were determined by western blot analysis for Bax, Bcl-2 and Cyclin D1 with GAPDH used as a standard. Each band is representative for three experiments.

FIGS. 11A-B show that SCNE is involved in the migration of HT29 colon cancer cells. Migration scratch assays were employed to study the role of SCNE (75 μg/ml) and nimbolide (10 μM) in migration of HT29 colon cancer cells. FIG. 11A shows SCNE (75 μg/ml) and Nimbolide (10 μM) were effective in inhibiting migration of HT29 cells. FIG. 11B shows that inhibition of migration was similarly effective with the treatment of nimbolide.

FIGS. 12A-E show that inhibition of transcription factor p65 nuclear localization and STAT3 phosphorylation and pro-inflammatory markers by SCNE and nimbolide in colon cancer cells HCT116 and HT29 cells. FIG. 12A shows HCT116 and HT29 cells were treated with SCNE and nimbolide for 48 hrs and were examined by immunofluorescent staining for p65 nuclear transport. SCNE and nimbolide blocked translocation of p65 protein to the nucleus. FIG. 12B show HCT116 and HT29 cells were treated with SCNE (40 and 75 μg/ml) for 48 hrs and the expression levels of pSTAT3, p65, IKKβ and GAPDH proteins were detected by western blot analysis. FIG. 12C show HCT116 and HT29 cells were treated with nimbolide (5 and 10 μM) for 48 hrs and the expression levels of pSTAT3, p65, IKKβ and GAPDH proteins were detected by western blot analysis. The data were obtained from 3 independent experiments. FIG. 12D show HCT116 and HT29 cells were treated with SCNE (40 and 75 μg/ml) for 48 hrs and the expression levels of COX1, COX2, IL-6, and TNF-α and GAPDH proteins were detected by western blot analysis. FIG. 12E show HCT116 and HT29 cells were treated with nimbolide (5 and 10 μM) for 48 hrs and the expression levels of COX1, COX2, IL-6, TNF-α and GAPDH proteins were detected by western blot analysis. GAPDH was used as a cell internal protein marker. The data were obtained from 3 independent experiments.

FIGS. 13A-B show inhibition of invasion by SCNE and nimbolide in colon cancer cells. FIG. 13A shows HCT116 and HT29 cells were treated with SCNE (40 and 75 μg/ml) for 48 hrs and the expression levels of MMP2, MMP9 and GAPDH proteins were detected by western blot analysis. Gelatin Zymogram assay showed concentration dependent inhibition in the proteolytic activity of MMP2 on SCNE treatment. FIG. 13B show HCT116 and HT29 cells were treated with nimbolide (5 and 10 μM) for 48 hrs and the expression levels of MMP2, MMP9 and GAPDH proteins were detected by western blot analysis. Gelatin Zymogram assay showed concentration dependent inhibition in the proteolytic activity of MMP2 on nimbolide treatment.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, the subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to the administering step.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be cancer or a hyperproliferative disorder.

As used herein, the term “inhibit” or “inhibiting” means decreasing tumor cell growth rate from the rate that would occur without treatment and/or causing tumor mass (e.g., cancer) to decrease. Inhibiting also include causing a complete regression of the tumor (e.g., cancer).

### Introduction

The risk for developing oral cancers is ever increasing world-wide as the global rise in tobacco use, alcohol consumption, and HPV exposure continues (Cancer Facts and Figures 2015). Oral squamous cell carcinomas (OSCC) comprise 90% of all oral cancers and represent the 6^{th} most common cancer in the world and the 8^{th} most common cancer in the United States (Cancer Facts and Figures 2015). Although conventional treatments have improved the 5-year survival rates for patients with early disease, patients with late-stage disease (stage III and stage IV) have a 5-year survival rate as low as 34% (Cancer Facts and Figures 2015). Furthermore, these statistics have not changed in nearly 40 years. Hence, prevention of OSCC initiation and progression is can be important to reducing the morbidity and mortality of this devastating disease.

Azadirachta indica, or neem, belongs to a family of trees related to mahogany; meliaceae (Hao F, Kumar S, Yadav N, Chandra D. Neem components as potential agents for cancer prevention and treatment. Biochim Biophys Acta; 1846:247-57). Neem is native to India, Myanmar, Bangladesh, Sri Lanka, Malaysia, and Pakistan and grows in tropical and semi-tropical regions around the world (Hao F, Kumar S, Yadav N, Chandra D. Neem components as potential agents for cancer prevention and treatment. Biochim Biophys Acta; 1846:247-57). The neem tree is a source of highly active liminoid terpenoids, collectively known as azadiractoids which are shown to have anti-cancer activity (Manikandan P, Ramalingam S M, Vinothini G, Ramamurthi V P, Singh I P, Anandan R, et al. Investigation of the chemopreventive potential of neem leaf subfractions in the hamster buccal pouch model and phytochemical characterization. Eur J Med Chem; 56:271-81). Previous studies of neem extracts in OSCC are limited to relatively impure ethanolic organic extracts from the neem leaf evaluated in the hamster cheek pouch carcinogenesis model where some activity is shown (Subapriya R, Kumaraguruparan R, Nagini S. Expression of PCNA, cytokeratin, Bcl-2 and p53 during chemoprevention of hamster buccal pouch carcinogenesis by ethanolic neem (Azadirachta indica) leaf extract. Clin Biochem 2006; 39: 1080-7; and Dasgupta T, Banerjee S, Yadava P K, Rao A R. Chemopreventive potential of Azadirachta indica (Neem) leaf extract in murine carcinogenesis model systems. J Ethnopharmacol 2004; 92: 23-36). Murine models of stomach and skin cancers also demonstrate efficacy of the neem leaf ethanolic extract which contains at least 35 biologically active compounds (Dasgupta T, Banerjee S, Yadava P K, Rao A R. Chemopreventive potential of Azadirachta indica (Neem) leaf extract in murine carcinogenesis model systems. J Ethnopharmacol 2004; 92: 23-36).

Aerial parts and seeds of the neem tree (Azadirachta indica) have been used as medicine to treat a number of human disease conditions. Neem has a rich use in Ayurvedic traditional medicine, and its folkoric use to treat pro-inflammatory conditions has led to the hypothesis that its anti-inflammatory potential could be useful in cancer prevention and treatment. Supporting this notion is the long history of traditional use of neem to treat acute and chronic inflammatory disease in India and Africa. Neem twigs, for instance, have long been used traditionally to maintain oral health and neem has been shown to have anti-bacterial, anti-fungal, and anti-ulcer properties. These observations lend credence to the idea that neem and its constituents may modulate cancer associated inflammation.

Organic solvent extracts of neem leaf have demonstrated anti-tumor effects in models of breast, prostate, and pancreatic cancer among others. Supercritical extract technology allows for better extraction of bioactive principals from natural compounds circumventing lability of active agents to heat or to solvent degradation. In some aspects, a supercritical CO₂ extract of neem leaf was used in the Examples disclosed herein and method of preparing said extract allows for better retention of innate volatiles. Neem is rich in volatile terpenoids (liminoids) which account for the bitter taste to the leaf, and as a class, are among the chief bioactive phytochemicals found in neem leaves. Few neem liminoids have been isolated in sufficient quantity to examine mechanism of action. Of the more common neem liminoids, the bulk of investigations have focused on nimbolide (5,7,4′-trihydroxy-3′,5′-diprenylflavone). Several studies have examined nimbolide for inhibition of growth from a number of cell lines, including neuroblastoma, leukemia, and melanoma. Nimbolide was also found to induce cell cycle alterations in breast and glioblastoma cell lines as well as modulate cell signaling pathways. Initial studies have reported that expression of VEGF and other metastasis enabling factors were inhibited in vitro. Supercritical leaf extracts are relatively safe to use and do not contain toxic compounds found in neem oil which is widely used as a natural insecticide. Since at least one supercritical extract of neem leaf has entered the commercial market as a constituent of many health products, its efficacy was examined in a range of colon cancer preclinical model systems, comparing its action with nimbolide.

Described herein is a supercritical CO₂ extract of neem (SCNE) that is highly pure such that the bioactive component, nimbolide, has been identified and the potential solute contaminants have been removed (Rodriguez-Solana R, Salgado J M, Dominguez J M, Cortes-Dieguez S. Comparison of Soxhlet, accelerated solvent and supercritical fluid extraction techniques for volatile (GC-MS and GC/FID) and phenolic compounds (HPLC-ESI/MS/MS) from Lamiaceae species. Phytochem Anal; 26:61-71). SCNE was evaluated for anti-proliferative effects in vitro and in vivo using cell based assays, a 4NQO-1 carcinogen model of OSCC, and mouse xenograft model of human OSCC. Effects on circulating cytokines and inflammatory and apoptotic markers are also described herein in addition to disruption of EGFR signaling which drives 90% of OSCC.

### Compositions

Disclosed herein are compositions that can be used with any of the methods disclosed herein. The compositions described herein can be a supercritical CO₂ neem extract (SCNE). Also disclosed herein are compositions comprising supercritical CO₂ neem extracts. In some aspects, SCNE can comprise nimbolide, nimbin and salinin. As disclosed herein, the concentrations of any of nimbolide, nimbin and salinin can vary. In some aspects, the SCNE can comprise at least 3 mg/g nimbolide. In some aspects, the SCNE can comprise at least 130 μg/g nimbin. In some aspects, the SCNE can comprise at least 200 μg/g salinin. In some aspects, the amount of the nimbolide present in the composition can be at least 3 mg/g; the amount of the nimbin present in the composition can be at least 130 μg/g nimbin; and the salinin present in the composition can be at least 200 μg/g. In some aspects, the concentrations of any of any of the components in the compositions described herein can vary.

In some aspects, the compositions described herein can further include one or more pharmaceutically acceptable excipients. Depending on the formulation, the inclusion of a pharmaceutically acceptable excipient can be optional. Examples of pharmaceutically acceptable excipients that can be used include but are not limited to dextrin/malto-dextrin or di-calcium phosphate, distilled water, saline, aqueous glucose solution, alcohol (e.g. ethanol), surfactants, propylene glycol, tween-80 and polyethylene glycol; and oily carriers such as various animal and vegetable oils, white soft paraffin, paraffin, wax, glucose, fructose, sucrose, maltose, yellow dextrin, malt dextrin, white dextrin, aerosol, microcrystalline cellulose, calcium stearate, magnesium stearate, sorbitol, stevioside, corn syrup, lactose, citric acid, tartaric acid, malic acid, succinic acid, lactic acid, L-ascorbic acid, dl-alpha-tocopherol, glycerin, propylene glycol, glycerin fatty ester, poly glycerin fatty ester, sucrose fatty ester, sorbitan fatty ester, propylene glycol fatty ester, acacia, carrageenan, casein, gelatin, pectin, agar, vitamin B group, nicotinamide, calcium pantothenate, amino acids, aerated or fumed silica, calcium salts, pigments, flavors and preservatives.

In some aspects, the SCNE can comprise one or more liminoids. In some aspects, the composition further comprises one or more tocopherols; and sesame oil. Examples of tocopherols include but are not limited to alpha-tocopherol, gamma-tocopherol, vitamin E (tocopherols) or Rosemary (Rosemarinus officinalis), CO₂ extract or any o anti-oxidant which is pharmaceutically accepted. In some aspects, the composition further comprises one or more tocopherols; sesame oil; and aerated or fumed silica.

In some aspects, the composition can comprise SNCo₂ extract: 75 mg; antioxidants such as, for example, Vitamin E or Rosemary (Rosemarinus officinalis) CO₂ extract: 10 mg and sesame oil: 415 mg.

In some aspects, the composition can comprise SNCO₂ extract: 50 mg; 582 mg dextrin/malto-dextrin or other carrier (e.g., di-calcium phosphate or any other pharmaceutical grade carrier); and 18 mg aerated or fumed silica.

In some aspects, the composition can comprise water extract obtained from Azadirachta indica leaf and used as a carrier for making a free-flowing powder of 50 mg Neem leaf extract obtained with supercritical CO₂ extraction as described herein.

In some aspects, the composition can comprise SNCO₂ extract: 2.28 g, peppermint (Mentha piperita) oil: 13.81 g, spearmint (Mentha spicata) oil: 9.26 g, clove bud (Syzigium aromaticum) CO₂ oil: 3.98 g, tween 80: 20.68 g. 1.25 g of the said blend was diluted in 98.75 g of Base. The base comprises: water: 73.5 g, Aloe Vera water (200×): 10 g, sorbitol: 10 g, glycerin: 5.9 g, ascorbic acid: 0.5 g, potassium sorbate: 0.1 g.

### Methods of Treatment

Disclosed herein, are methods of treating cancer in a subject, the method comprising: (a) identifying a subject in need of treatment; and (b) administering to the subject a therapeutically effective amount of a supercritical CO₂ neem extract (SCNE). In an aspect, the SCNE can comprise nimbolide, nimbin and salinin. Also, disclosed herein, are methods of treating cancer in a subject, the method comprising: (a) identifying a subject in need of treatment; and (b) administering to the subject a therapeutically effective amount of a composition comprising a therapeutically effective amount of a supercritical CO₂ neem extract (SCNE). In some aspects, SCNE can comprise nimbolide, nimbin and salinin. The concentrations of any of nimbolide, nimbin and salinin can vary. In some aspects, the SCNE can comprise at least 3 mg/g nimbolide. In some aspects, the SCNE can comprise at least 130 μg/g nimbin. In some aspects, the SCNE can comprise at least 200 μg/g salinin. In some aspects, the amount of the nimbolide present in the composition can be at least 3 mg/g; the amount of the nimbin present in the composition can be at least 130 μg/g nimbin; and the salinin present in the composition can be at least 200 μg/g. In some aspects, the concentrations of any of any of the components in the compositions described herein can vary. In some aspects, the composition can further comprise a pharmaceutically acceptable excipient. In an aspect, the subject can be a human.

Disclosed herein, are methods of reducing at least one inflammatory cytokine in serum of a subject in need thereof. In some aspects, the method can comprise administering to the subject a composition comprising a therapeutically effective amount of a supercritical CO₂ neem extract (SCNE). In an aspect, the SCNE can comprise nimbolide, nimbin and salinin. The concentrations of any of nimbolide, nimbin and salinin can vary. In some aspects, the SCNE can comprise at least 3 mg/g nimbolide. In some aspects, the SCNE can comprise at least 130 μg/g nimbin. In some aspects, the SCNE can comprise at least 200 μg/g salinin. In some aspects, the amount of the nimbolide present in the composition can be at least 3 mg/g; the amount of the nimbin present in the composition can be at least 130 μg/g nimbin; and the salinin present in the composition can be at least 200 μg/g. In some aspects, the composition can further comprise a pharmaceutically acceptable excipient. In an aspect, the subject can be a human. In some aspects, the at least one inflammatory cytokine can be IFN-γ, IFN-β, TNF-α, IL-6 or IL-1. In some aspects, the subject can have, be suspected of having or be diagnosed with oral cancer. In some aspects, the administration to the subject of a composition comprising a therapeutically effective amount of a supercritical CO₂ neem extract, wherein SCNE comprises nimbolide, nimbin and salinin can reduce at least one inflammatory cytokine in serum of the subject, wherein the subject has, is suspected of having or has been diagnosed with oral cancer. In some aspects, the at least one inflammatory cytokine can be IL-6 or TNF-α. In some aspects, IFN-γ, IFN-β, TNF-α, IL-6 or IL-1 can be reduced after the administration of a therapeutically effective amount of a supercritical CO₂ neem extract described herein in a subject that has, is suspected of having, or has been diagnosed with oral cancer, In some aspects, the subject's serum can have increased levels of at least one inflammatory cytokine when compared to a reference sample before the administration of the composition comprising therapeutically effective amount of a supercritical CO₂ neem extract. In some aspects, the methods can further comprise determining the level of at least one inflammatory cytokine in one or more cells of a subject before the administration of the composition comprising a therapeutically effective amount of a supercritical CO₂ neem extract. In some aspects, the level of at least one inflammatory cytokine can be higher when compared to a reference sample.

Disclosed herein, are methods of reducing inflammation in a subject in need thereof. The method can comprise administering to the subject a composition comprising a therapeutically effective amount of a supercritical CO₂ neem extract (SCNE). In some aspects, the SCNE can comprise nimbolide, nimbin and salinin. The concentrations of any of nimbolide, nimbin and salinin can vary. In some aspects, the SCNE can comprise at least 3 mg/g nimbolide. In some aspects, the SCNE can comprise at least 130 μg/g nimbin. In some aspects, the SCNE can comprise at least 200 μg/g salinin. In some aspects, the amount of the nimbolide present in the composition can be at least 3 mg/g; the amount of the nimbin present in the composition can be at least 130 μg/g nimbin; and the salinin present in the composition can be at least 200 μg/g. In some aspects, the composition can further comprise a pharmaceutically acceptable excipient. In an aspect, the subject can be a human. In some aspects, the subject has been diagnosed with oral cancer or colon cancer prior to the administering step. In some aspects, the inflammation can be reduced by decreasing the expression of one or more of IFN-γ, IFN-β, TNF-α, IL-6, IL-1, NF-KB, STAT3, COX1 or COX2.

Disclosed herein, are methods of treating a hyperproliferative disorder in a subject in need thereof. The method can comprise administering to the subject a composition comprising a therapeutically effective amount of a supercritical CO₂ neem extract (SCNE). In an aspect, the SCNE can comprise nimbolide, nimbin and salinin. The concentrations of any of nimbolide, nimbin and salinin can vary. In some aspects, the SCNE can comprise at least 3 mg/g nimbolide. In some aspects, the SCNE can comprise at least 130 μg/g nimbin. In some aspects, the SCNE can comprise at least 200 μg/g salinin. In some aspects, the amount of the nimbolide present in the composition can be at least 3 mg/g; the amount of the nimbin present in the composition can be at least 130 μg/g nimbin; and the salinin present in the composition can be at least 200 μg/g. In some aspects, the composition can further comprise a pharmaceutically acceptable excipient. In an aspect, the subject can be a human. In some aspects, the subject has been diagnosed with a need for treatment of the hyperproliferative disorder prior to the administering step. In an aspect, the hyperproliferative disorder can be cancer. In some aspects, the hyperproliferative disorder can be oral cancer or colon cancer.

Disclosed herein, are methods of suppressing expression of NFkB and cycloxygenase in a subject in need thereof. The method can comprise administering to the subject a composition comprising a therapeutically effective amount of a supercritical CO₂ neem extract (SCNE). In some aspects, the SCNE can comprise nimbolide, nimbin and salinin. The concentrations of any of nimbolide, nimbin and salinin can vary. In some aspects, the SCNE can comprise at least 3 mg/g nimbolide. In some aspects, the SCNE can comprise at least 130 μg/g nimbin. In some aspects, the SCNE can comprise at least 200 μg/g salinin. In some aspects, the amount of the nimbolide present in the composition can be at least 3 mg/g; the amount of the nimbin present in the composition can be at least 130 μg/g nimbin; and the salinin present in the composition can be at least 200 μg/g. In some aspects, the composition can further comprise a pharmaceutically acceptable excipient. In an aspect, the subject can be a human. In some aspects, the subject has been diagnosed with a need for suppressing the expression of NFkB and cyclooxygenase prior to the administering step. In some aspects, the subject has been diagnosed with a need for treatment of a disorder of uncontrolled cellular proliferation prior to the administering step. In some aspects, the methods can further comprise the step of identifying a subject in need of treatment of a disorder of uncontrolled cellular proliferation. In some aspects, the disorder of uncontrolled cellular proliferation can be a cancer. In some aspects, the cancer can be an oral cancer.

Disclosed herein, are methods of suppressing expression of NFkB and cyclooxygenase in at least one cell. In some aspects, the method can comprise the step of contacting at least one cell with an effective amount of a supercritical CO₂ neem extract (SCNE). In some aspects, SCNE can comprise nimbolide, nimbin and salinin. The concentrations of any of nimbolide, nimbin and salinin can vary. In some aspects, the SCNE can comprise at least 3 mg/g nimbolide. In some aspects, the SCNE can comprise at least 130 μg/g nimbin. In some aspects, the SCNE can comprise at least 200 μg/g salinin. In some aspects, the amount of the nimbolide present in the composition can be at least 3 mg/g; the amount of the nimbin present in the composition can be at least 130 μg/g nimbin; and the salinin present in the composition can be at least 200 μg/g. In some aspects, the composition can further comprise a pharmaceutically acceptable excipient. In some aspects, the at least one cell can be a human cell. In some aspects, the contacting step can be via administration to a subject. In some aspects, the subject has been diagnosed with a need for treatment of a disorder of uncontrolled cellular proliferation prior to the administering step. In some aspects, the subject has, is suspected of having or has been diagnosed with oral cancer. In some aspects, the uncontrolled cellular proliferation can be oral cancer.

Disclosed herein, are methods of modifying epidermal growth factor receptor signaling (EGFR) activity in a subject. The method can comprise administering to the subject a composition comprising a therapeutically effective amount of a supercritical CO₂ neem extract (SCNE). In some aspects, the SCNE can comprise nimbolide, nimbin and salinin. The concentrations of any of nimbolide, nimbin and salinin can vary. In some aspects, the SCNE can comprise at least 3 mg/g nimbolide. In some aspects, the SCNE can comprise at least 130 μg/g nimbin. In some aspects, the SCNE can comprise at least 200 μg/g salinin. In some aspects, the amount of the nimbolide present in the composition can be at least 3 mg/g; the amount of the nimbin present in the composition can be at least 130 μg/g nimbin; and the salinin present in the composition can be at least 200 μg/g. In some aspects, the composition can further comprise a pharmaceutically acceptable excipient. In an aspect, the subject can be a human. In some aspects, the subject has been diagnosed with a need for modifying EGFR signaling activity. In some aspects, the modifying can be inhibiting. In some aspects, SCNE can inhibit EGFR signaling activity. In some aspects, the subject has been diagnosed with a need for treatment of a disorder of uncontrolled cellular proliferation prior to the administering step. In some aspects, the methods further comprise the step of identifying a subject in need of treatment of a disorder of uncontrolled cellular proliferation. In some aspects, the disorder of uncontrolled cellular proliferation can be oral cancer.

Disclosed herein, are methods of inducing apoptosis of a cell in a subject in need thereof. The method can comprise administering to the subject a composition comprising a therapeutically effective amount of a supercritical CO₂ neem extract (SCNE). In some aspects, the SCNE can comprise nimbolide, nimbin and salinin. The concentrations of any of nimbolide, nimbin and salinin can vary. In some aspects, the SCNE can comprise at least 3 mg/g nimbolide. In some aspects, the SCNE can comprise at least 130 μg/g nimbin. In some aspects, the SCNE can comprise at least 200 μg/g salinin. In some aspects, the amount of the nimbolide present in the composition can be at least 3 mg/g; the amount of the nimbin present in the composition can be at least 130 μg/g nimbin; and the salinin present in the composition can be at least 200 μg/g. In some aspects, the composition can further comprise a pharmaceutically acceptable excipient. In an aspect, the subject can be a human. In some aspects, subject has been diagnosed with a need for treatment of a disorder of uncontrolled cellular proliferation prior to the administering step. In some aspects, the methods can further comprise the step of identifying a subject in need of treatment of a disorder of uncontrolled cellular proliferation. In some aspects, the disorder of uncontrolled cellular proliferation can be a cancer. In some aspects, the cancer can be colon cancer.

The compositions described herein can be formulated to include a therapeutically effective amount of a supercritical CO₂ neem extract described herein. In an aspect, the supercritical CO₂ neem extract can comprise nimbolide, nimbin and salinin. The concentrations of any of nimbolide, nimbin and salinin can vary. In some aspects, the SCNE can comprise at least 3 mg/g nimbolide. In some aspects, the SCNE can comprise at least 130 μg/g nimbin. In some aspects, the SCNE can comprise at least 200 μg/g salinin. In some aspects, the amount of the nimbolide present in the composition can be at least 3 mg/g; the amount of the nimbin present in the composition can be at least 130 μg/g nimbin; and the salinin present in the composition can be at least 200 μg/g. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of cancer.

The concentration or amounts of each of nimbolide, nimbin and salinin can vary in a single composition. In some aspects, the SCNE can comprise at least 3 mg/g nimbolide. In some aspects, the SCNE can comprise at least 130 μg/g nimbin. In some aspects, the SCNE can comprise at least 200 μg/g salinin. In some aspects, the amount of the nimbolide present in the composition can be at least 3 mg/g; the amount of the nimbin present in the composition can be at least 130 μg/g nimbin; and the salinin present in the composition can be at least 200 μg/g. The concentration or amount of each component can vary according to many factors, for example, the particular the type and severity of the cancer as well as the type of formulation.

In some aspects, the SCNE can comprise one or more liminoids. In some aspects, the SCNE further comprises azadirachtin A, azadirachtin B, and deacetylsalinin. In some aspects, the SCNE further comprises small amounts of one or more of azadirachtin A, azadirachtin B, and deacetylsalinin. In an aspect, “small amounts” can refer to amounts that can be at the limit of detection.

In some aspects, the compositions described herein can further comprise one or more tocopherols; and sesame oil. Examples of tocopherols include but are not limited to alpha-tocopherol, gamma-tocopherol, vitamin E (tocopherols) or Rosemary (Rosemarinus officinalis), CO₂ extract or any other naturally occurring anti-oxidant which is pharmaceutically accepted. In some aspects, the composition further comprises one or more tocopherols; sesame oil; and aerated or fumed silica.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions are administered to a subject (e.g., a human patient) already with or diagnosed with cancer in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the cancer is delayed, hindered, or prevented, or the cancer or a symptom of the cancer is ameliorated. One or more of the symptoms can be less severe. Recovery can be accelerated in an individual who has been treated.

Disclosed herein, are methods of treating a patient with cancer. The cancer can be any cancer. In some aspects, the cancer can be oral cancer or colon cancer. In some aspects, the cancer can be a primary or secondary tumor. In an aspect, the subject has been diagnosed with cancer prior to the administering step.

The compositions described herein can be formulated to include a therapeutically effective amount of supercritical CO₂ neem extract as described herein alone or in combination with one or more therapeutic agents or therapies or treatment regimens. In an aspect, the one or more therapeutic agents or therapies or treatment regimens can be chemotherapy or radiotherapy. In an aspect, SCNE can be contained within a pharmaceutical formulation. In an aspect, the pharmaceutical formulation can be a unit dosage formulation. The compositions described herein can be formulated in a variety of combinations. The particular combination of SCNE with one or more chemotherapeutic agent or radiotherapy can vary according to many factors, for example, the particular the type and severity of the cancer.

The therapeutically effective amount or dosage of the supercritical CO₂ neem extract as described herein used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the cancer symptoms), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the compositions can be administered once a day; once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly. In an aspect, the compositions described herein can be administered twice to three times a day. In some aspects, the compositions described herein can be administered twice to three times a day for two to three to four weeks (or longer). In some aspects, the compositions described herein can be administered two to three times a day. In some aspects, the compositions described herein can be administered two to three times a day for two, three or four weeks.

Dosages of SCNE can be in the range of 50 mg to 1000 mg/day. In an aspect, SCNE can be administered in a dosage ranging from about 50 mg to 1000 mg/day. In an aspect, the dosage of SCNE can be 25, 50, 75, 100, 125, or 150 mg/day or any amount in between. In some aspects, the dosage of SCNE can be more than 150 mg/day. In some aspects, the dosage of SCNE can be 160, 170, 180, 190, 200, 250, 300, 350, 400, 450, 500, 550, 600, 650, 700, 750, 800, 850, 900, 950 or 1000 mg/day or any amount in between. In an aspect, the compositions described herein can be in the form of a capsule. In some aspects, the capsule can be administered orally one, two or three times a day. In some aspects, the compositions can be administered orally one, two or three times a day for 21 or 28 days. In some aspects, the compositions can be administered orally two or three times a day for 21 or 28 days. In some aspects, the compositions can be administered for 1 month, 2 months, 3 months, 4 months, 5 months or 6 months or longer. In some aspects, the can be administered orally two or three times a day for 6 months.

In some aspects, the total effective amount of the compositions as disclosed herein can be administered to a subject as a single dose, either as a bolus over a relatively short period of time, or can be administered using a fractionated treatment protocol in which multiple doses are administered over a more prolonged period of time.

In some aspects, the compositions described herein can be administered in conjunction with other therapeutic modalities to a subject in need of therapy. The present compounds can be given to prior to, simultaneously with or after treatment with other agents or regimes. For example, supercritical CO₂ neem extract as described herein can be administered in conjunction with standard therapies used to treat cancer. In an aspect, any of the compositions described herein can be administered or used together with chemotherapy or radiotherapy.

### Pharmaceutical Compositions

As disclosed herein, are pharmaceutical compositions, comprising supercritical CO₂ neem extract as described herein and a pharmaceutical acceptable carrier described herein. In some aspects, SCNE can be formulated for oral administration. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

In some aspects, the compositions disclosed herein can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

In some aspects, the compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used. In an aspect, the composition can be in a form comprising a capsule.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). In some aspects, the pharmaceutically acceptable excipient can be dextrin/malto-dextrin or di-calcium phosphate. In some aspects, the excipient can vary depending on the formulation. In some aspects, the excipient can be optional. In some aspects, the pharmaceutically acceptable excipient and carrier can be selected from the group consisting of distilled water, saline, aqueous glucose solution, alcohol (e.g. ethanol), surfactants, propylene glycol, tween-80 and polyethylene glycol; and oily carriers such as various animal and vegetable oils, white soft paraffin, paraffin, wax, glucose, fructose, sucrose, maltose, yellow dextrin, malt dextrin, white dextrin, aerosol, microcrystalline cellulose, calcium stearate, magnesium stearate, sorbitol, stevioside, corn syrup, lactose, citric acid, tartaric acid, malic acid, succinic acid, lactic acid, L-ascorbic acid, dl-alpha-tocopherol, glycerin, propylene glycol, glycerin fatty ester, poly glycerin fatty ester, sucrose fatty ester, sorbitan fatty ester, propylene glycol fatty ester, acacia, carrageenan, casein, gelatin, pectin, agar, vitamin B group, nicotinamide, calcium pantothenate, amino acids, aerated or fumed silica, calcium salts, pigments, flavors and preservatives. The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral administration. In an aspect, the composition can be administered orally. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes SCNE dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). In an aspect, the compositions can be for the formulation of a liquid (e.g., a mouthwash). The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. In an aspect, the compositions can be packaged in a container comprising multiple doses in a liquid form.

In an aspect, a pharmaceutical composition comprises SCNE; and optionally, a pharmaceutical acceptable carrier. The SCNE can comprise nimbolide, nimbin and salinin. In some aspects, the SCNE can comprise at least 3 mg/g nimbolide. In some aspects, the SCNE can comprise at least 130 μg/g nimbin. In some aspects, the SCNE can comprise at least 200 μg/g salinin. In some aspects, the amount of the nimbolide present in the composition can be at least 3 mg/g; the amount of the nimbin present in the composition can be at least 130 μg/g nimbin; and the salinin present in the composition can be at least 200 μg/g. In an aspect, the pharmaceutical composition can be formulated for oral administration. In an aspect, the composition can be formulated as a capsule or a liquid.

In some aspects, the compositions described herein can be provided in a therapeutically effective formulation for oral administration comprising: SNCO₂ extract: 75 mg; antioxidants such as, for example, Vitamin E (tocopherols) or Rosemary (Rosemarinus officinalis) CO₂ extract: 10 mg and sesame oil: 415 mg. Said composition can be put into a soft gel capsule of 500 mg. In some aspects, the capsules can be administered to a patient two times a day for a total of 50-1000 mg to four times a day for a total of 1000 mg of Neem leaf extract per day as an active drug.

In some aspects, the compositions described herein can be provided in a therapeutically effective formulation for oral administration comprising: SNCO₂ extract: 50 mg; 582 mg dextrin/malto-dextrin or other carrier (e.g., di-calcium phosphate or any other pharmaceutical grade carrier); and 18 mg aerated or fumed silica. A free-flowing powder can be prepared and encapsulate in “00” size hard gelatin or vegetarian capsule. In an aspect, the treatment regimen can be 1 capsule twice a day to 3 capsules thrice a day in 21 or 28-days cycle for 6 cycles. In some aspects, the compositions described herein can be provided in a therapeutically effective formulation for oral administration comprising: water extract obtained from Azadirachta indica leaf and used as a carrier for making a free-flowing powder of 50 mg Neem leaf extract obtained with supercritical CO₂ extraction as described herein. Said composition can be administered two times a day to achieve a therapeutic dose of 150 mg to four times a day for a total of 1000 mg Neem leaf CO₂ extract as described herein.

In some aspects, the compositions described herein can be provided in a therapeutically effective formulation as a mouthwash comprising: SNCO₂ extract: 2.28 g, peppermint (Mentha piperita) oil: 13.81 g, spearmint (Mentha spicata) oil: 9.26 g, clove bud (Syzigium aromaticum) CO₂ oil: 3.98 g, tween 80: 20.68 g. 1.25 g of the said blend was diluted in 98.75 g of Base. The base comprises: water: 73.5 g, Aloe Vera water (200×): 10 g, sorbitol: 10 g, glycerin: 5.9 g, ascorbic acid: 0.5 g, potassium sorbate: 0.1 g. The formulation described herein is a liquid formulation that can be administered 1-3 times a day, 20 ml each time, to achieve a therapeutic dose of 150-1000 mg Neem Leaf CO₂ extract as described above. The mouthwash formulation can be used to treat or prevent oral cancer.

In some aspects, a fine-sized nano-particle CO₂, in which a minimum of 10% of the extract are nanoparticles, can be prepared by increasing the velocity and passing the particle through a micro-jet or nozzle, can be administered orally 2 or 3 times a day to achieve therapeutically effective dose of 100-500 mg Neem leaf CO₂ extract nanoparticle as described above, instead of 1000 mg of ordinary CO₂ extract.

The formulations disclosed herein can be prepared using the nano-particle extracts described herein.

### Articles of Manufacture

The composition described herein can be packaged in a suitable container labeled, for example, for use as a therapy to treat cancer or any of the methods disclosed herein. Accordingly, packaged products (e.g., sterile containers containing the composition described herein and packaged for storage, shipment, or sale at concentrated or ready-to-use concentrations) and kits, including at least SCNE as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compounds can be ready for administration (e.g., present in dose-appropriate units), and may include a pharmaceutically acceptable adjuvant, carrier or other diluent. Alternatively, the compounds can be provided in a concentrated form with a diluent and instructions for dilution.

### EXAMPLES

### Example 1

Oral Squamous Cell Carcinoma Growth Inhibition by Neem (Azadirachta indica) Extract In Vivo: Disruption of the inflammation cascade, reduction in tumor occurrence and volume in oral squamous cell carcinoma by treating with a natural Neem leaf extract.

The leaves and bark of the Neem tree (Azadirachta indica) have been used in traditional Ayurvedic medicine for centuries to treat oral maladies. The experiments described herein tested the hypothesis that use of this Neem leaf extract can prevent initiation and/or progression of OSCC. The anti-cancer potential of the Neem leaf extract was tested in in vitro and in vivo platforms. OSCC cell lines (SCC4, Cal27, HSC3) were treated with the leaf extract at various time points while markers of inflammation, invasion, and proliferation were analyzed. The preventive effects of SCNE were also assessed in ectopic xenograft mouse models and a carcinogen induced mouse model of oral cancer. Treatment with the Neem leaf extract inhibited OSCC cell proliferation, reduced levels in markers of inflammation in OSCC cells. The Neem leaf extract reduced wound closure, showing inhibition of metastasis. Xenografted nude mice showed significant reduction of OSCC tumor occurrence and reduced tumor growth. The Neem leaf extract also significantly reduced tumors and tongue dysplasias in a 4NQO-1 mouse oral carcinogen model. In both cancer animal models, the Neem Leaf extract depressed circulating inflammatory cytokines. Chemopreventive effects of SCNE were also examined on the inhibition and prevention of OSCC both in vitro and in vivo. The results show a marked decrease in tumor proliferation, reduction of inflammatory markers and circulating cytokines, which strongly support the potential of SCNE as prevention agent in a standalone regime or in combination with standard frontline therapies to improve patient outcomes.

### Materials and Methods

Reagents. Supercritical CO₂ neem extract was provided by Nisarga Ltd, Sartara, India. Leaves from organically grown neem trees are processed with supercritical CO₂. Supercritical extracts have the advantage of replacing organic solvents with excellent solvency and no organic residues remain resulting in a highly pure neem extract (SCNE) (Lindskog M A, Nelander H, Jonson A C, Halvarsson T. Delivering the promise of SFC: a case study. Drug Discov Today; 9: 1607-12). Stock solutions of 50 mg/ml in 100% DMSO were used in vitro. Nimbolide was purchased from Biovision (#2356) and dissolved in 100% DMSO to a stock of 1 mg/ml. SCNE diets were manufactured by Harlan Teklad to deliver 200 mg/kg SCNE—SNCE was dissolved in corn oil and homogenously mixed with the remaining diet ingredients and formed into pellets. Celecoxib (PZ0008, Sigma, USA) was dissolved in 100% DMSO to a stock of 1 mg/ml.

Human OSCC Cell Lines and MTT assay. SCC4 and Cal27 oral cancer cells are obtained from ATCC and maintained in DMEM supplemented with 10% fetal bovine serum and 1% penicillin/streptomycin at 37° C. with 5% CO₂. For the SCC4 cells, hydrocortisone was provided at 400 ng/ml in the completed media. For the cell treatments, SCNE (Nisarga Ltd.) was used and applied at different concentrations (1-400 μg/m) for 8 h, 24 h, or 48 h to 75% confluent cells. Doses to be tested will bracket the IC₅₀ₛ for each cell line. Control cells will receive DMSO. Nimbolide was applied at different concentrations (1-100 μM) for 8 h, 24 h, or 48 h to 50% confluent cells. For the celecoxib treatments cell were treated at different concentrations (1-200 ug/ml) for 8 h, 24 h, or 48 h. Cells were cultured overnight in complete media, serum-starved for 24 hr, and treated vehicle, SCNE, nimbolide, or Celecoxib as described herein. Subsequently, 10 μl of 12 mM MTT (Life Technologies; Carlsbad, Calif.) solution was added to each well, incubated for 4 hr at 37° C., and neutralized with DMSO. Absorbance was measured at 540 nm and percent viability was calculated.

Gelatinase Zymogram. Gelatinase zymography was performed in 10% SDS polyacrylamide gel in the presence of 0.1% gelatin under non-reducing conditions. Colon cancer cells were grown in 96 well plates. Culture media 200 μl was collected from each well (pool of 3×) and concentrated to final volume 20 μl. Culture media (20 μl) were mixed with sample buffer and loaded for SDS-PAGE without boiling. Following electrophoresis the gels were washed twice in 1× Zymogram renaturing Buffer containing Triton X-100 (Thermo Scientific, MA) for 1 hr at Room Temperature to remove SDS. The gels were then incubated in 1× Zymogram Developing Buffer containing the substrate (Thermo Scientific, MA) for 48 hrs at 37° C. and stained with 0.5% Coomassie Blue R250 in 50% methanol and 10% glacial acetic acid for 60 min and destained. Upon renaturation of the enzyme, gelatinases digest gelatin in the gel and give clear bands against an intensely stained background. Protein standards and 2% fetal bovine serum (positive control) were run concurrently and appropriate molecular weights were determined by plotting the relative mobilities of known proteins (PMID 28440509).

Cell migration assay. SCC4, Cal27, and HSC3 cells were cultured in 96-well plates in complete growth medium. A scratch was performed using a WoundMaker and visualized using the IncuCyte ZOOM real time imaging system (Essen BioScience, MI, USA). Cells were treated with 20 or 60 μg/ml SCNE or 10 or 50 μM Nimbolide and imaged at 3 hr intervals for 72-120 hr to monitor cell migration and wound healing.

Protein Expression. Cellular protein extracts will be prepared and proteins quantified as described previously. Briefly, cells will be washed twice with 1× PBS, collected by scraping and centrifuged at 4° C. at 300 g for 6 min. The pellet will be resuspended in 250 μl of Buffer A (10 mM Tris-HCl pH 7.8, 10 mM KCl, 1.5 mM MgCl₂, 1 tab protease inhibitor and water) and incubated on ice for 10 min. The samples will then be homogenized at 15,000 rpm for 45 s on ice and then centrifuged at 4,600 g for 5 min at 4° C. The supernatent will be removed and stored in −80° C. as the cytosolic protein fraction. The collected pellet will be resuspended in 100 μl of Buffer B (210 mM Tris-HCl pH 7.8, 420 mM KCl, 1.5 mM MgCl₂, 20% glycerol, 1 tab of protease inhibitor and water) followed by gentle agitation at 4° C. for 30 min and centrifugation at 10,000 g for 10 min at 4° C. The supernatant will be collected and stored at −80° C. as the nuclear protein fraction. Fifty micrograms of either cytosolic or nuclear protein fractions will be separated by SDS-PAGE (12% gels) and transferred to PVDF membranes (Bio-Rad, USA). The membranes will be probed with the primary antibodies (Cell Signalling, USA) NFkB p65 (8242S), STAT3 (8768S) pSTAT3 (9131S), COX1 (9896S), COX2 (12282S), EGFR (4267S), pEGFR (4404S), ERK1/2 (T202/Y204-9101S), AKT (9272S), pAKT (9271S) followed by horseradish peroxidase-conjugated anti-rabbit (7074S). GAPDH (2118S) or Topo IIα (12286S) will be used to ensure equal protein loading. The immunoreactive bands will be visualized on ChemiDoc Touch (Bio-Rad, USA) using chemiluminescent substrate (Clarity ECL, Bio-Rad). Bands will be quantitated with ChemiDoc software (Bio-Rad).

Animals. Six week-old female athymic nude mice (Harlan, Indianapolis, Ind.) were used in a laminar air-flow cabinet under pathogen-free conditions. They were provided with a 12 hr light/dark schedule at controlled temperature and humidity with food and water ad libitum. Mice were acclimated for one week prior to study initiation.

OSCC Mouse Xenograft Models. Mice were injected subcutaneously in the right flank with 3×10⁶ HSC3 or 10×10⁶ SCC4 or 6×10⁶ Cal27 cells in 0.2 ml of sterile PBS as previously described. Mice were placed on AIN76A synthetic diet for 24 hours. Then SCNE diet (200 mg/kg) was placed into the Neem treatment group, then control group remained on the AIN76A. For the HSC3 animal groups, nimbolide was injection by intraparentaerally for 5 consecutive days, starting at day 10 post tumor injection, at 5 mg or 20 mg Nimbolide/kg mouse. Tumor volumes were calculated by the elliptical formula: ½(Length×Width²) (Jensen M M, Jorgensen J T, Binderup T, Kjaer A. Tumor volume in subcutaneous mouse xenografts measured by microCT is more accurate and reproducible than determined by 18F-FDG-microPET or external caliper. BMC Med Imaging 2008; 8:16). Blood was drawn at termination and serum isolated for cytokine analysis.

CBA carcinogen induced Oral Cancer Model. Twenty CBA mice were placed on AIN76A or 200 mg/kg SCNE diet and given 4-NQO-1 (Sigma) at 100 μg/ml in their drinking water. The mice were kept on the 4NQO-1 water for 8 weeks, followed by 4 weeks of regular water. At 12 weeks, blood was drawn at terminations for serum cytokine analysis and tongues excised and fixed in formalin.

Immunohistochemistry. The formalin fixed tongue were paraffin embedded and sliced at 1 microns. Immunostaining was done following method previously published (PMID 27167203) using the following antibodies (Abcam: PCNA ab18197; Ki-67 ab16667; c-Met ab51067).

Cytokine and Chemokine Assay. Serum cytokine/chemokine profiles were taken at termination and stored at −80° C. until analysis using the Bio-Plex Pro group 1 mouse cytokine 23-plex assay kit and analyzed with the Bio-Plex 200 Luminex-based multiplex analysis system (Bio-Rad, Hercules, Calif.).

Statistical Analysis. Statistical analysis was performed using GraphPad Prism4 (San Diego, Calif.). Cell viability and migration assays were analyzed by one-way ANOVA and Bonferroni's post-hoc test. Statistical analyses of tumor growth were made using analysis of variance with repeated measures with Bonferroni's post-hoc test. A p value less than 0.05 was considered statistically significant.

### Results

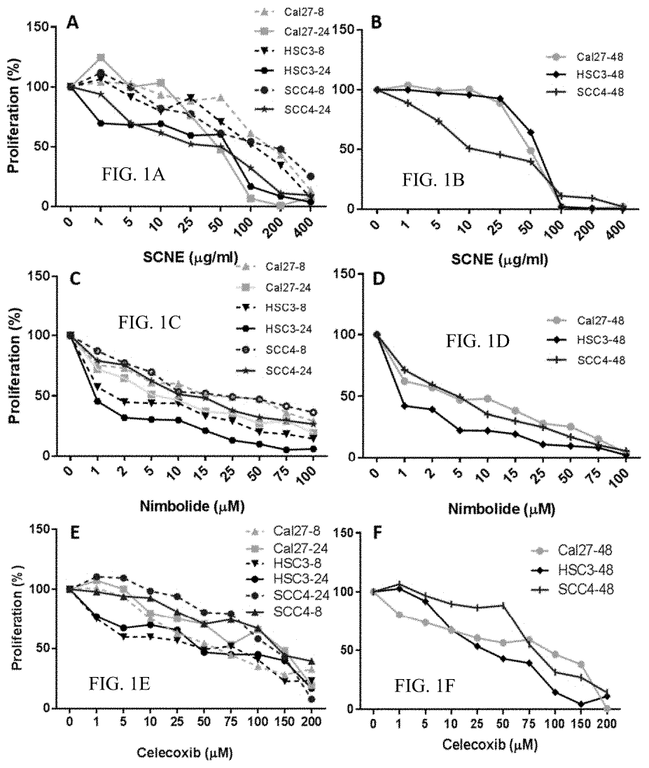

SCNE and Nimbolide inhibit oral squamous cancer cell growth. There are previous reports of some anti-cancer of Neem extracts, mainly alcohol derived, as well as, effects with Nimbolide, a single compound from in Neem leaf extracts. However, to date there are no reports investigating the anti-cancer potential of a solvent free, hydrophobic and hydrophilic constituent containing, supercritical CO₂ extract of Neem leaves. Utilizing SCNE and Nimbolide, three different OSCC human cell lines were treated at various doses and three time points (8 h, 24 h, 48 h) to determine cytotoxic concentrations and IC50 (FIG. 1A-D). The SCNE reduced cell growth in a dose and time dependent manner with an IC50 of 50 μg/ml of SNCE at the time points tested (FIG. 1A, 1C) and 15 μM Nimbolide for three time points (FIG. 1B, 1D). Next, the SCNE and Nimbolide cytotoxic effects were compared to a standard non-steroidal anti-inflammatory, Celecoxib (FIG. 1E-F). At 8 h and 24 h treatments the IC50 for Celecoxib was 75 μM as well as a similar IC50 at 48 h was markedly less than Nimbolide. These results show that SCNE and Nimbolide have cytotoxic effects on OSCC cell lines similar to or slightly higher than standard NSAIDs. From this data, 20 and 60 μg/ml SCNE and 10 and 50 μM were chosen to further understand the mechanisms of action.

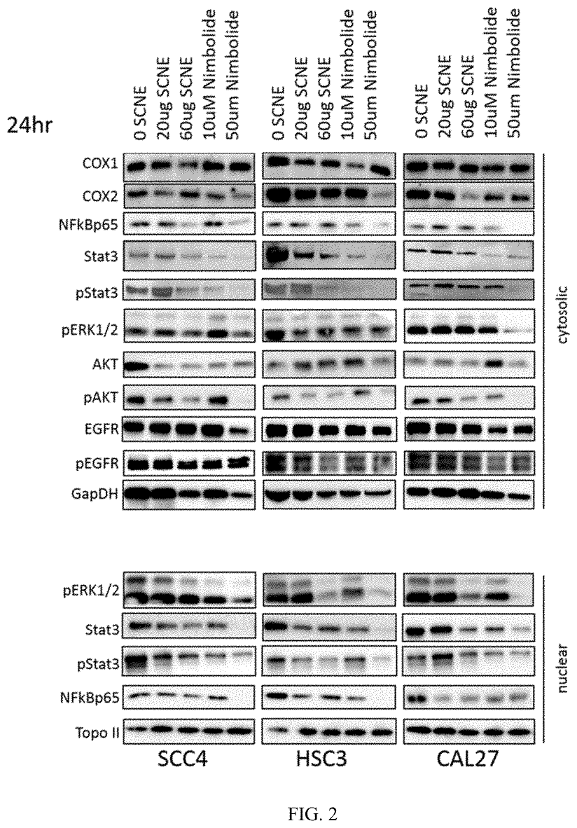

SCNE and Nimbolide down regulate inflammatory mediators. To elucidate the mechanism(s) of action of SCNE and Nimbolide, three OSCC cell lines were treated with 20 μg, 60 μg/ml SCNE and 10 mM, 50 mM Nimbolide and analyzed for cytosolic and nuclear protein fractions (PMID 27167203). Reports have shown that inflammatory markers, such as NFkB, cyclooxygenases, as well as cellular proliferators STAT3, AKT, and ERK1/2 are elevated in OSCC. Treatment with SCNE or Nimbolide moderately decreases COX2 levels with minimal effect on COX1 (FIG. 2) but a modest effect on NFkBp65 at higher doses of SCNE and Nimbolide was observed. Both SNCE and Nimbolide showed drastic down regulation of pSTAT3, pAKT and pERK1/2. However, little change in EGFR and pEGFR was observed in response to treatments. In the nuclease, SCNE and Nimbolide showed stronger reduction on NFkBp65 and pERK1/2. A similar trend in STAT3 and pSTAT3 reduction was also observed in response to SCNE and Nimbolide. These results confirm the anti-inflammatory and anti-proliferative potency of SCNE and Nimbolide in OSCC.

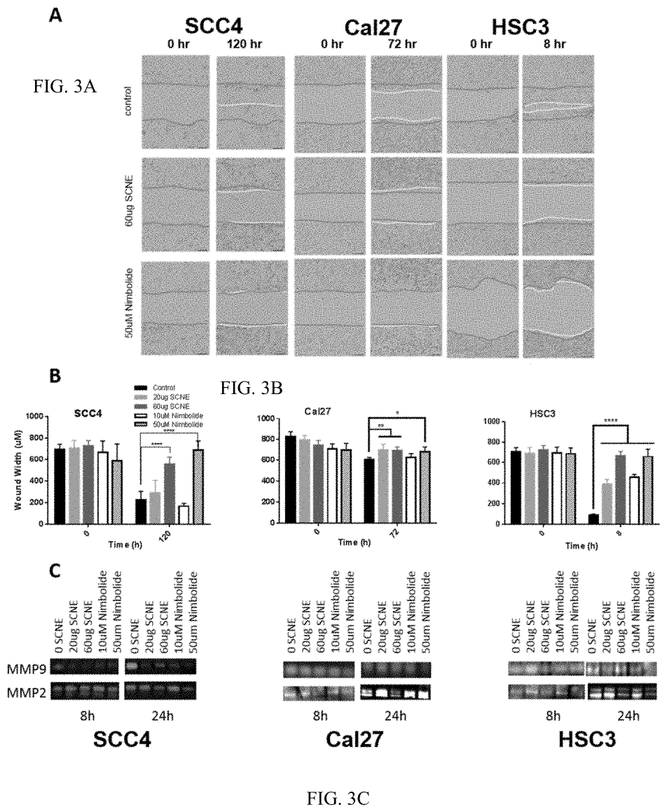

SCNE and Nimbolide inhibit in vitro cell migration. The in vitro results suggests a strong cytotoxic effect on OSCC through down-regulation of inflammatory mediators and cellular proliferation markers. To better understand the anticancer potential of SCNE and Nimbolide, their anti-metastatic effects were assessed. Utilizing a wound healing assay, it was observed that both SCNE and Nimbolide significantly reduce cell migration (FIG. 3A-B). The highly mobile cell line HSC3 reduced the wound (90%) in 8 h, however, SCNE and Nimbolide inhibited this closure, with less than 10% closure. In the less mobile line, SCC4, SCNE and Nimbolide halted cell migration across the wound in a similar fashion, although this occurred after 120 h (FIGS. 3A-B). Cal27 cells are not very mobile, however, SNCE and Nimbolide did inhibit the modest cell migration compared to the untreated group. Given these strong results in perturbing cellular migration, the effects of SCNE and Nimbolide were assessed on two metalloprotease proteins MMP2 and MMP9. In the highly mobile HSC3 OSCC cell line, SNCE and Nimbolide reduced MMP2 activity with slight reduction in MMP9. In the SCC4 cell line, MMP9 was drastically reduced SCNE and Nimbolide, with little change in MMP2. The non-mobile Cal27 cell line had modest reduction in MMP2 activity with SCNE treatment and MMP9 treatment with Nimbolide. Taken together, the in vitro results suggest a strong anti-tumor effect of SCNE and Nimbolide through down regulation of proliferative markers, reduction in inflammatory markers, and reduced cellular migration.

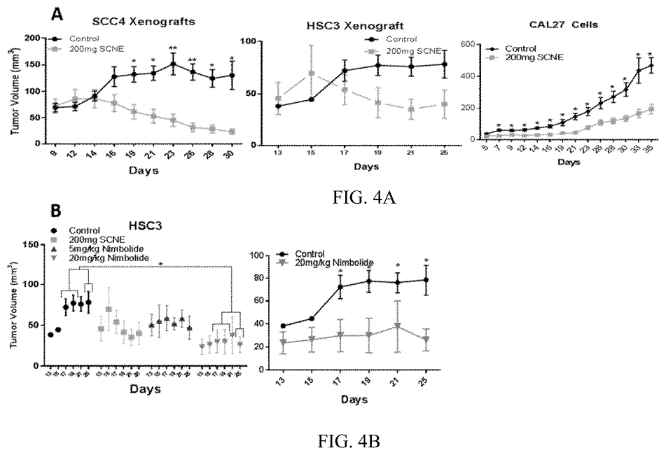

SNCE and Nimbolide inhibits OSCC derived tumor growth in mice. To corroborate the results from the OSCC experiments, the same three cell lines in xenografted mouse models (FIG. 4) were used. 200 mg/kg SCNE was incorporated into the diet to deliver the therapeutic neem extract and a synthetic AIN73A diet as control. In one cell line, HSC3, 5 mg or 20 mg Nimbolide was injected by IP for 5 consecutive days. At termination the SCNE diet significantly reduced SCC4 tumor volume (81%) and Cal27 (49%) volume compared to the untreated controls (FIG. 4A). SCNE did reduce HSC3 tumor volume (49%), however, high variance did not produce a significant result. The 20 mg/kg Nimbolide treatment did significantly reduce tumor volume (69%) and the 5 mg/kg showed a moderate reduction (40%) in volume (FIG. 4B). Body weight data were comparable between experimental arms (data not shown). These data affirm both SCNE and nimbolide have profound anti-tumor activity in vivo.

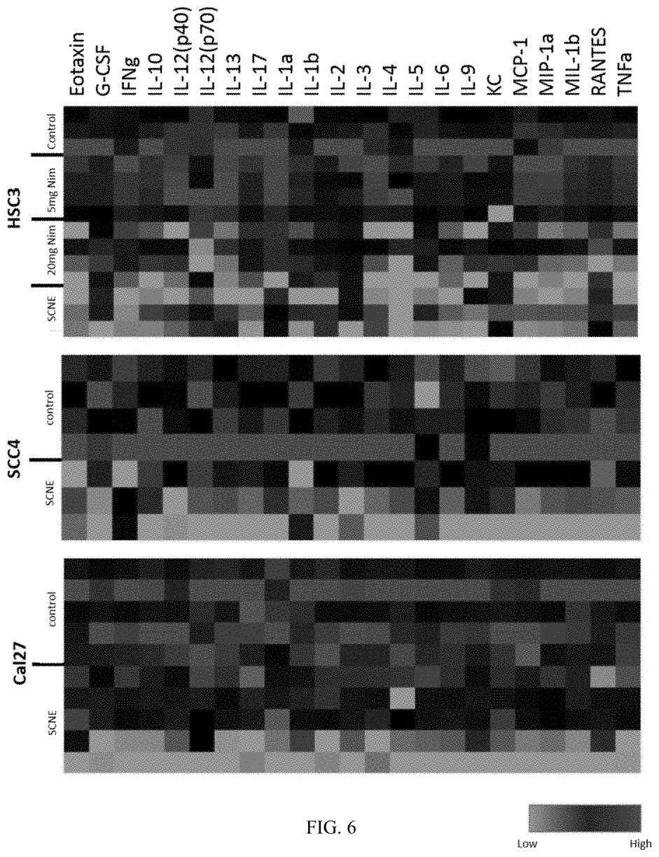

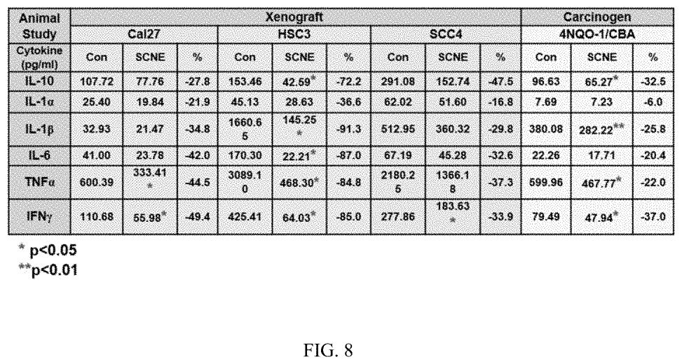

SCNE and Nimbolide reduce serum level inflammatory cytokines in xenografted mice. The serum from the above mice used to investigate the effects of SCNE on the circulating inflammatory cytokine population (FIG. 8) was analyzed. The extract strongly reduced IL-1b, TNFα, IFNγ, and IL-6 serum levels. For IL-1α, there was a moderate reduction in the level of IL-1α identified in the xenografted mice. A similar pattern was observed for IL-10 but the stronger reduction occurred in the HSC3 tumor bearing mice. Many other cytokines from these animals were examined (FIG. 6) and overall SCNE treatment produced a different profile compared to control. Combined with tumor volume reduction, the data suggests that SCNE can reduce tumor burden and deleterious inflammatory cytokines.

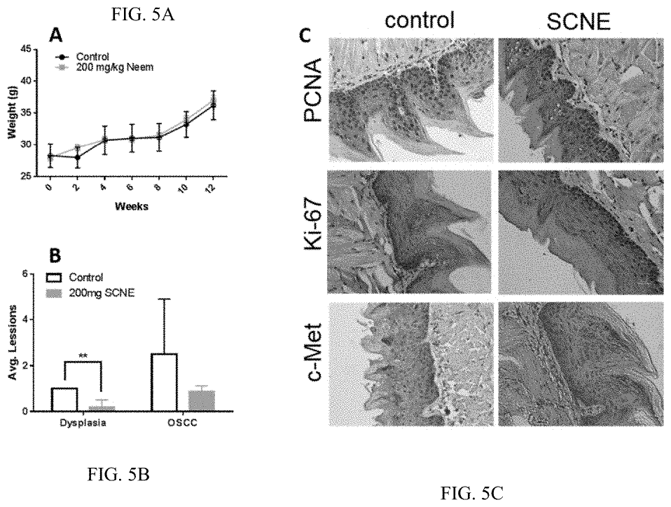

SCNE inhibits tumor growth in 4NQO-1 mouse model of OSCC. To further validate the in vivo anti-cancer potential of SCNE, a 4-NQO-1 induced tongue OSCC model in the CBA strain was established. The 4NQO-1 (50 μg/ml) was administered in the drinking water for 8 weeks and then regular water replaced it for another 4 weeks. The control mice were fed ad libitum AIN73A diet and the treatment group the same 200 mg/kg SCNE diet for the entire 12 week study. To assess the palatability of the diet, mice weight was measured every 2 weeks and the mice showed no difference in weight gain (FIG. 5A). At termination, the tongues pathobiology was examined for any dysplasias and/or tumors. The SCNE significantly reduced early stage dysplasia compared to controls and 66% reduction in OSCC tumors (FIG. 5B). The expression levels of proliferative markers in the tongues was also characterized by immunohistochemistry and it was found that SCNE reduced PCNA, Ki-67, and c-Met protein levels (FIG. 5C).

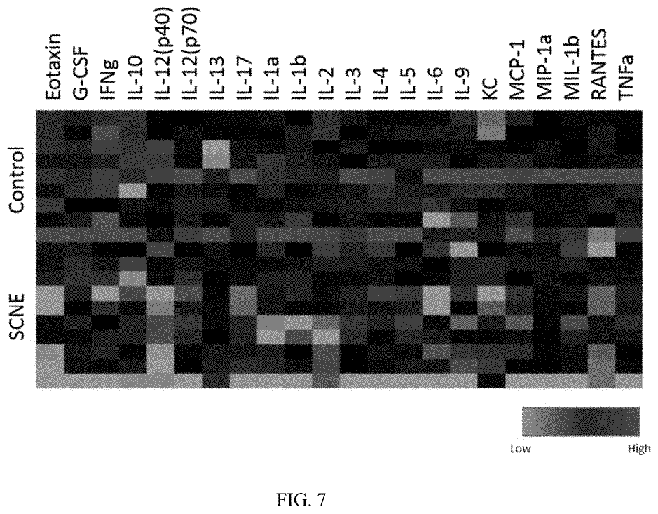

SCNE reduces serum inflammatory cytokines in a carcinogen induced mouse model of OSCC. In addition to the pathobiology of these mice, the serum circulating cytokine inflammatory population was examined. The SCNE significantly reduced IFNγ, IL-1β and TNFα levels in these animals (FIG. 8). Two other cytokines IL-6 (30%) and IL-1α (25%) levels were reduced after 12 weeks of SCNE diet consumption. Many other cytokines from these animals were examined (FIG. 7) and overall SCNE and Nimbolide treatment produced a different profile compared to control. This pattern follows similar reductions in the xenograft animal studies further suggesting the anti-cancer and anti-inflammatory effects of SCNE.

### Discussion

The anti-cancer effects of SCNE in OSCC was shown to occur through downregulation of key tumor proliferating markers and reduction in inflammatory modulators. The Neem leaf extract reduced wound closure, showing inhibition of metastasis. Xenografted nude mice showed significant reduction of OSCC tumor occurrence and reduced tumor growth. The Neem leaf extract also significantly reduced tumors and tongue dysplasia in a 4NQO-1 mouse oral carcinogen model. In both cancer animal models, the Neem Leaf extract depressed circulating inflammatory cytokines.

Currently, OSCC cancers are on the rise and in the clinic most chemotherapies are of the standard variety with very little second line options. To improve this scenario, new avenues to treatment with novel agents and combination therapies could overcome this problem. For instance, COX2 expression has been shown to be elevated in OSCC cases and contribute to radioresistance.

The chemo preventive effects of SCNE, known for its biomedicinal properties, were examined on the inhibition and prevention of OSCC, both in vitro and in vivo. The data described herein show a marked decrease in tumor proliferation, reduction of inflammatory markers, and circulating cytokines. With the dearth of clinical treatment options for OSCC frontline and second line therapies, this extract may be used as a prevention agent in a standalone regime or in combination with standard frontline therapies to improve patient outcomes and/or resistant recurrent tumors in relapsed patients.

### Example 2

A Supercritical CO₂ Extract of Neem Leaf (A. indica) and its Bioactive Liminoid, Nimbolide, Suppress Colon Cancer in Preclinical Models by Modulating Pro-inflammatory Pathways.

To explore a role of neem in CRC, human colon cancer cell lines HCT116 and HT29 cells were treated with purified Super Critical Neem Extract (SCNE) or nimbolide. SCNE treatment showed a dose dependent inhibition of CRC cell proliferation and an increase in apoptosis. It was found that treatment of both SCNE and nimbolide showed anti-inflammatory effect by poor nuclear localization of p65, decreased protein expression of transcriptional factor phosphorylated STAT3 and pro inflammatory cytokines COX1, COX2, IL-6 and TNF-α in CRC cells. Western blots and Zymogram results showed anti-invasive effect by decreased expression of MMP2 and MMP9 proteins in CRC cells on treatment with SCNE. Overall, these data confirms a potential anti-cancer effect of SCNE, reducing cell proliferation, inflammation, migration, and invasion and inducing apoptosis in human Colon cancer cells.

### Materials and methods

Cell lines and Cell Culture. Human colon cancer cell lines HCT116 and HT29 were obtained from American Type Culture Collection (ATCC). Both of these cell lines were cultured in McCoy's 5A medium supplemented with pyruvate, vitamins, amino acids, antibiotics and 10% fetal bovine serum. Rat colon normal epithelial cell line IEC6 was obtained from American Type Culture Collection [IEC6] (ATCC® CRL-1592™). IEC6 Cells were cultured in Dulbecco's modified Eagle's medium (DMEM) with 4 mM L-glutamine, 1.5 g/L sodium bicarbonate, 4.5 g/L glucose, 0.1 Unit/ml bovine insulin and 10% fetal bovine serum. The cultures were maintained at 37° C. in a humidifier incubator with 5% CO₂. To determine dose-dependent changes in protein and gene expression, cells were treated with different concentrations of Supercritical Extract Neem Extract, SCNE (Nisarga, India) and nimbolide (Biovision, USA) or an equal volume of Dimethyl Sulfoxide (DMSO) as a vehicle for different time period as needed.

Cell Viability assay. Colorectal cancer cells, HCT116 and HT29 as well as normal Rat Colon cells IEC-6 cells, were plated in 96-well plates, next day the cells were serum starved for 24 hrs and treated with SCNE (0-150 μg/mL) and nimbolide (1-100 μM) for 48 h and 72 h. After treatment, cell viability was measured by MTT [3-(4,5-Dimethylthiazol-2-yl)-2, 5-Diphenyltetrazolium Bromide] assay (Sigma Aldrich, MO) according to the manufacturer's instructions. Briefly, MTT (5 mg/mL) was added and plates were incubated at 37° C. for 4 h before dimethyl sulfoxide was added to each well. Finally, the absorbance of each well was read at a wavelength of 540 nm using a plate reader (Molecular Devices, Sunnyvale, Calif., USA). The results were expressed as a percentage of surviving cells over non treated cells.

Migration Scratch Assay. Migration assays were performed using the IncuCyte ZOOM system (Essen BioScience, Inc., MI) to measure migration of the colon cancer cells without and with SCNE and nimbolide treatment. A wound was created in confluent cells in each well (10 replicates) and the ingrowth of the cells were calculated over a definite time period by masking wound boundaries at 0 h against untreated cells, to measure wound closure.

Western Blot Analysis. HCT-116 and HT-29 cells were grown to confluent in 100 mm culture dishes. Cells were serum starved for 24 hrs. Next day, the cells were treated with different doses of SCNE and nimbolide against vehicle (DMSO) for 48 hrs at 37° C. Whole cell lysates were prepared with RIPA lysis buffer. Nuclear Protein extraction: Non-treated and treated HCT-116 and HT-29 cells were kept on ice for 10 min with low salt Lysis buffer (10 mM HEPES, 10 mM KCl, 1 mM EDTA), then scrapped and spun down. Pellet was collected and 50-100 μl of high-salt lysis buffer was added and incubated on ice for 30 min with intermediate vortex. The tubes were spun down and nuclear protein was collected from the supernatant. Protein concentration was determined using Pierce BCA Protein Assay Kit (Thermo Scientific, MA).

Equal amounts of protein were separated on 7.5%, 10% and 12% SDS_PAGE. Then, proteins were transferred to Immun-Blot PVDF membranes for protein Blotting (Bio Rad, CA) and blocked in 5% non-fat milk in Tris-buffered saline with 0.1% Tween-20 (TBST) for 1 h at room temperature. Antibodies against COX1 (Cell Signaling Technology, MA), COX2 (Cell Signaling Technology, MA), Bcl-2 (Abcam, MA), Bax1(Abcam, MA), TNF-α COX2 (1:500; Cell Signaling Technology, MA), IL-6 (Cell Signaling Technology, MA), Cyclin D1 (Cell Signaling Technology, MA), p65 (Abcam, MA), IKKβ (Abcam, MA), MMP2 (Abcam, MA), MMP9(Abcam, MA), pSTAT3 (Y705) (Cell Signaling Technology, MA), Topoisomerase, (Abcam, MA), and GAPDH (Sigma-Aldrich, MO) were diluted in 5% skim milk. Horseradish peroxidase-conjugated goat anti-rabbit (Abcam, MA) antibody was used as a secondary antibody.

Gelatinase Zymogram: Gelatinase zymography was performed in 10% SDS polyacrylamide gel in the presence of 0.1% gelatin under non-reducing conditions. Colon cancer cells were grown in 96 well plates. Culture media 200 μl was collected from each well (pool of 3×) and concentrated to final volume 20 μl. Culture media (20 μl) were mixed with sample buffer and loaded for SDS-PAGE without boiling. Following electrophoresis the gels were washed twice in 1× Zymogram renaturing Buffer containing Triton X-100 (Thermo Scientific, MA) for 1 hr at Room Temperature to remove SDS. The gels were then incubated in 1× Zymogram Developing Buffer containing the substrate (Thermo Scientific, MA) for 48 hrs at 37° C. and stained with 0.5% Coomassie Blue R250 in 50% methanol and 10% glacial acetic acid for 60 min and destained. Upon renaturation of the enzyme, the gelatinases digest the gelatin in the gel and give clear bands against an intensely stained background. Protein standards and 2% fetal bovine serum (positive control) were run concurrently and appropriate molecular weights were determined by plotting the relative mobilities of known proteins (25997494).

Immunofluorescence Microscopy: Quiescent human CRC cells were grown in multiwell plastic chamber slides and treated with SCNE or nimbolide for 48 hrs. At the termination of the study time, cells were washed twice with ice-cold PBS and fixed in methanol at −20° C. for 5 min. After a brief rinse, cells were blocked with 0.1% BSA in PBS and then stained with p65 using indirect immunofluorescence. Alexa Fluor 594 donkey anti-rabbit antibody served as secondary antibody (Thermo Fisher Scientific, MA). Stained cells were washed with PBS, mounted with prolong Gold antifade reagent with DAPI (Thermo Fisher Scientific, MA) mounted with coverslips, viewed and photographed using Zeiss LSM710 Confocal microscope (Carl Zeiss Microscopy, LLC, NY).

### Results

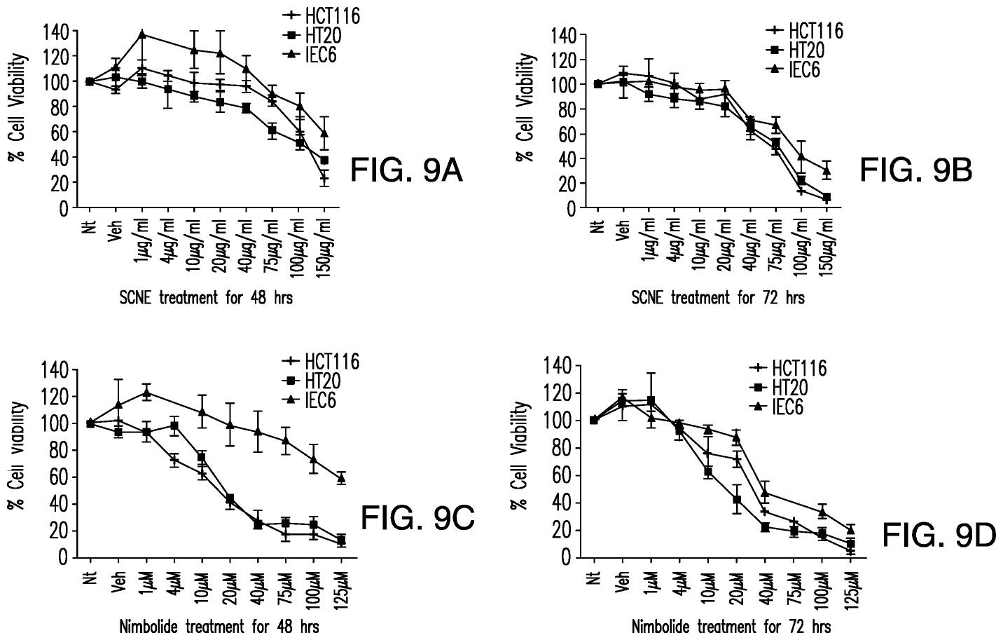

SCNE and Nimbolide inhibited proliferation in human CRC cells. To evaluate the effect of SCNE on human CRC cells, cell viability was analyzed using MTT assay. To investigate whether SCNE and Nimbolide have direct effects on CRC cells, the proliferation inhibition caused by SCNE and Nimbolide was tested in HCT-116 and HT-29 human CRC cell lines as well as normal rodent colon cell line IEC-6 by the MTT assay. Treatment of HCT-116 and HT29 with different concentrations of SCNE and Nimbolide for 48 and 72 h, resulted in a decrease in the cell viability (FIG. 9). Normal rodent colon cell line growth (IEC6) was not affected by SCNE as well as nimbolide. These results show that SCNE could inhibit CRC cell viability in a concentration and time dependent manner. The IC₅₀ for the SCNE and Nimbolide were determined to be <75 μg/ml and <10 μM, respectively. In further experiments, CRC cells were treated with SCNE dose of 40 μg/ml and 75 μg/ml and nimbolide dose of 5μM and 10 μM for 48 hrs.

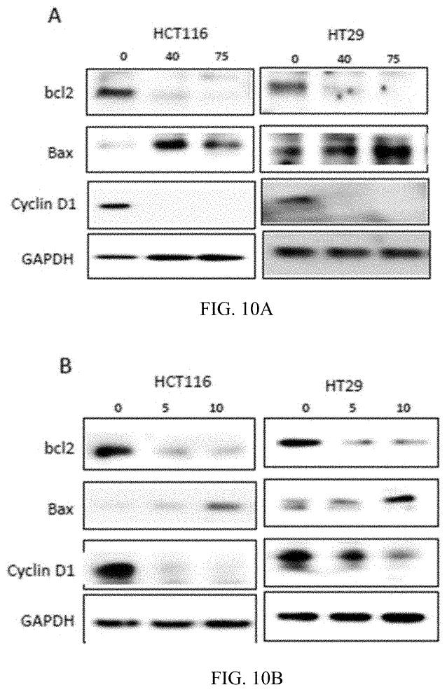

SCNE induced apoptosis in CRC cells. Suppression of apoptosis during carcinogenesis is thought to play a central role in the development and progression of some cancers. Tumor cells can acquire resistance to apoptosis by the expression of either anti-apoptotic proteins such as Bcl-2 or by down-regulation of pro-apoptotic proteins such as Bax. To figure out the relationship between induction of apoptosis and the expression of their regulatory proteins by SCNE treatment, the expression of apoptosis regulatory proteins was investigated. SCNE resulted in a decreased expression of anti-apoptotic marker Bcl-2 protein and upregulation of pro-apoptotic marker Bax protein in both HCT116 and HT29 cells (FIG. 10A) which was similar to nimbolide treatment in HCT116 and HT29 cells (FIG. 10B).

Cyclin D1 is a protein required for progression through the G1 phase of the cell cycle. Overexpression of Cyclin D1 has been shown to correlate with early cancer onset and tumor progression. CRC cell lines showed higher expression of Cyclin D1 protein which was significantly reduced upon treatment with SCNE and Nimbolide for 48 hrs (FIG. 10).

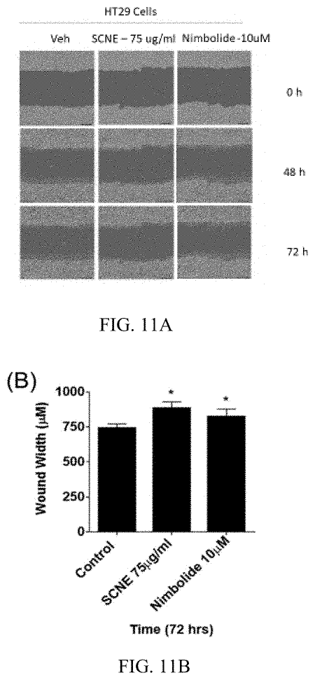

SCNE inhibited migration of human CRC cells. It was then determined whether the cell anti-proliferative and apoptotic activity of the SCNE and Nimbolide might translate into possible inhibition of cell migration, forecasting a potential inhibition of invasion. To test this, migration assays were conducted using the IncuCyte ZOOM system to measure migration of CRC cells without and with SCNE and Nimbolide treatment. It was found that SCNE and Nimbolide, both inhibited wound closure after 72 h treatment in a dose-dependent manner in HT-29 human colon cancer cells (FIG. 11).

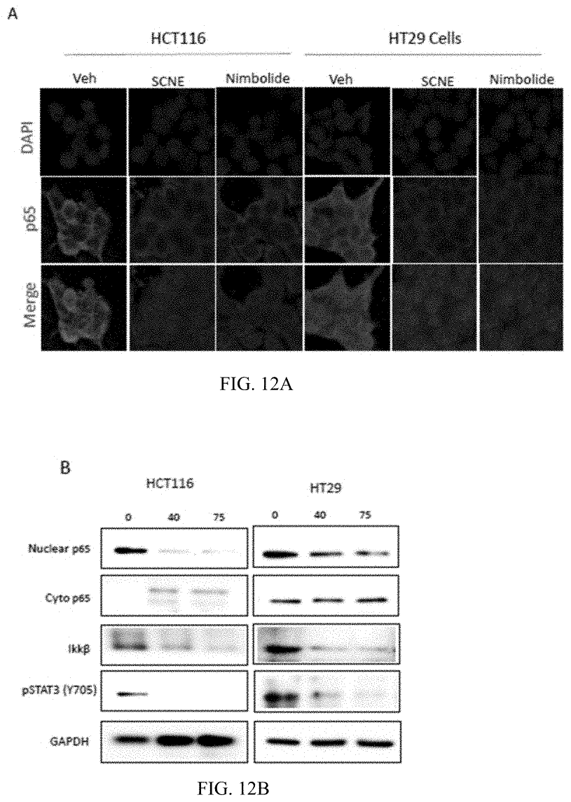

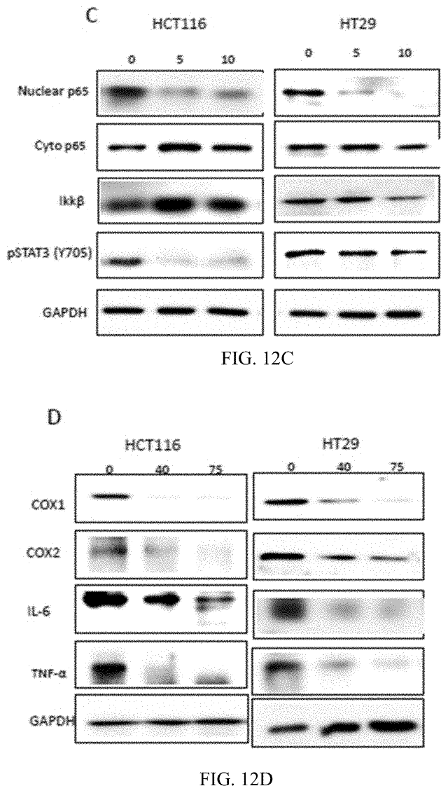

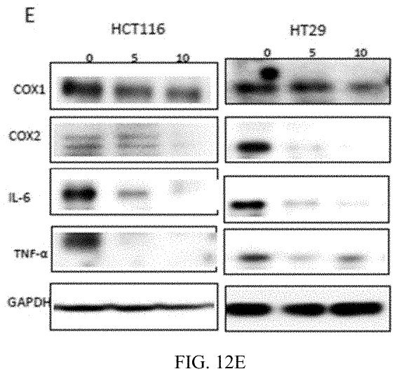

SCNE have anti-inflammatory activity—NF-κB/IL-6/STAT3 expression in CRC Cells. NF-κB and STAT3 regulate the expression of a large number of genes involved in inflammation. To determine whether SCNE and Nimbolide treatment on CRC cell lines HCT-116 and HT-29 exhibited an anti-inflammatory effect, the impact of SCNE and Nimbolide treatment on CRC cell lines was assessed. Here, it was found that treatment of HCT-116 and HT-29 cells for 48 h with the IC₅₀ of SCNE and Nimbolide resulted in reduced translocation of p65 from cytoplasm to the nucleus (FIG. 12A, 12B, 12C) and decreased expression of pSTAT3 protein expression, showing a loss of available NF-κB and STAT3 transcriptional factors to the nucleus.

IL-6 and TNF-α are pro-inflammatory cytokines and are highly expressed during cancer. The results from the experiments described herein show that treatment of CRC cell lines with SCNE and Nimbolide remarkably decreased the expression of IL-6 and TNF-α protein (FIG. 12D, 12E).

COX1 is constitutively expressed in human colon tissues whereas tumorigenic factor such as COX2 has been involved in colon tumorigenesis. The results showed that treatment of SCNE and Nimbolide decreased the protein expression of both COX1 and COX2 in CRC cell lines (FIG. 12D, 12E). Taken together, these data showed that SCNE and Nimbolide have an anti-inflammatory effect on CRC cell lines.

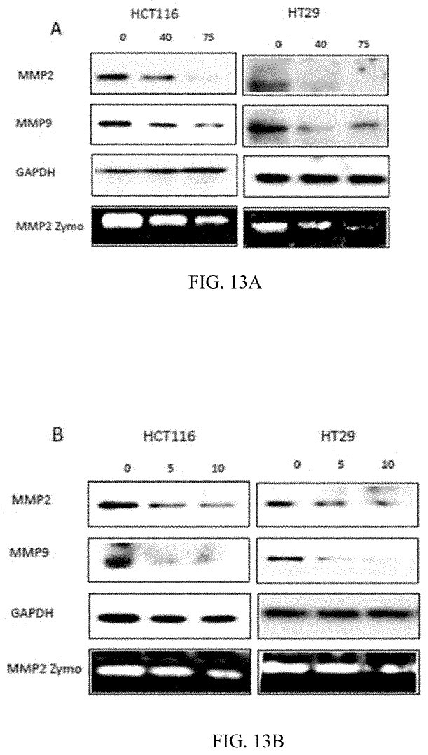

SCNE inhibited invasion in human CRC cells. MMPs are involved in invasion, migration, metastasis and tumorigenesis. Among the many MMPs that have been identified, gelatinases, especially MMP-2 (gelatinase A) and MMP-9 (gelatinase B), are thought to a play a key role in degradation of type IV collagen and gelatin, the two main components of ECM. To examine that metalloproteinase was responsible, gelatinase Zymography of HCT116 and HT29 cells treated with SCNE and Nimbolide against vehicle was performed. HCT116 and HT29 cells demonstrated strong secretion of MMP2 in serum free media which was inhibited by SCNE and Nimbolide after 48 h of treatment (FIG. 13A, 13B). More MMP2 expression in the medium directly correlated to more digestion of gelatin in the gel resulting in a clear band in untreated cells. Western blot analysis of human CRC cell lines showed higher expression of MMP2 and MMP9 in untreated cells. Treatment of colorectal cancer cells with SCNE and Nimbolide significantly decreased the expression of MMPs.

### Discussion

Given the high mortality rate as a result of colon cancer and the significant morbidity, apparent toxicity and poor response rates of current chemotherapeutic regimens, there has been a big push to identify novel therapeutic modalities with fewer toxicity profiles. Targeted therapies against VEGF (bevacizumab) or against EGFR (cetuximab) are now commonly used as treatments for CRC. However, patients develop resistance to such treatments; thus, new strategies are required to replace or complement current therapies. Dietary alterations can lead to widespread differences in the risks and incidences of several types of cancers. Additionally, the long term consumption of natural products present in fruits and spices, with proven safety, favoring their use in cancer chemoprevention. The approach of tumor prevention using safe and nontoxic novel plant derived agents has been fortified by many scientists. Plentiful natural products have been investigated for their potential use as anticancer agents. Neem is one such natural herb with demonstrable anti-cancer properties and is a source of several limonoids, which are a class of oxygenated triterpenes called tetranorterpenoids. These limonoids are responsible for the anti-tumor effects of neem leaf extract (NLE). However, the underlying mechanism of its inhibition of colorectal cancer cell proliferation and metastasis remains to be elucidated.

As described herein, it was investigated whether SCNE can exert anticancer activity against CRC via modulation of proinflammatory pathways in CRC cells and in animal model. Cell viability was assessed using an MTT assay in the absence or presence of various concentrations of SCNE. It was found that SCNE inhibited proliferation, migration and induced apoptosis in CRC cells. Therefore, the anti-proliferative and anti-migratory effects of SCNE observed in the present study were dependent on its cancer-preventive effects. Nimbolide caused cell cycle arrest at G1/S phase. Evidently, nimbolide was found to reduce cyclin A level, which is required for colon cancer cells to proceed through S phase, hence inducing cell cycle arrest and resulting in inhibition of cell growth. Antiapoptotic proteins and proapoptotic proteins regulate the level of activation of caspase3. Nimbolide treatment decreased the expression of antiapoptotic proteins (Bcl-xL, Bcl-2, survivin, caspase inhibitor molecules) and increased the expression of proapoptotic proteins (cytochrome c, Bax, Bad, Bid, cleaved caspases) in prostate cancer cells, which is similar to the results disclosed herein with SCNE treatment in CRC cells. The overexpression of proteins associated with cell survival and cell proliferation has been shown to contribute to tumor development. Down-regulation of the expression of proteins involved with cell survival and proliferation may contribute to the decreased growth of colon cancer cells. The observed antiproliferative and apoptosis inducing properties of SCNE are in agreement with those observed by others in leukemia and in colon cancer.

In the present study, the results described herein show that SCNE can inhibit the expression of proteins involved in tumor invasion, metastasis, and angiogenesis (MMP-9, MMP-2) which further supports the role of SCNE against CRC. Fucoidan inhibited cell growth, migration and sphere formation by suppressing the PI3K/Akt/mTOR pathway and reducing the expression of MMP-2 in human HT-29 colon cancer cells. Magnolol significantly downregulated matrixmetalloproteinase-9(MMP9) expression, an enzyme critical to tumor invasion and also inhibited nuclear factor-kB (NF-kB) transcriptional activity showing that its role in suppresses tumor invasion by inhibiting MMP-9 through the NF-kB pathway in human breast cancer. (24226295). Nimbolide inhibited proliferation, induced apoptosis, and suppressed NF-κB activation and NF-κB-regulated tumorigenic proteins in CRC cells. Nimbolide injected intraperitoneally after tumor inoculation, significantly decreased the volume of CRC xenografts. The limonoid-treated xenografts exhibited significant down-regulation in the expression of proteins involved in tumor cell survival (Bcl-2, Bcl-xL, c-IAP-1, survivin, Mcl-1), proliferation (cMyc, cyclin D1), invasion (MMP-9, ICAM-1), metastasis (CXCR4), and angiogenesis (VEGF).

It was found that HCT-116 and HT-29 colon cancer cells exhibited constitutive NF-κB and that SCNE suppressed this activation. It was shown that nimbolide inhibited inducible and constitutive NF-κB activation in leukemia and multiple myeloma cells. Constitutive NF-κB has been found to be important for the survival and proliferation of various tumor cell types by regulating the expression of proteins involved in tumor development. Therefore, it is likely that nimbolide exerts its inhibitory effects on tumor survival and growth by inactivating NF-κB. One of the possible mechanisms for the constitutive activation of NF-κB in tumor cells is through IKK activation. Avicins were found to be potent inhibitors of TNF-α induced NF-κB and to slow the accumulation of the p65 subunit of NF-κB in the nucleus. Avicin G treatment decreased the expression of NF-κB regulated proteins such as iNOS and COX-2. Other studies showed that pretreating cells with triterpenoids for 24 hours significantly reduced the induction of NF-κB mediated through TNF-α. Cycloartane triterpenoids from Cimicifuga dahurica suppressed the expression of cdc2 and COX-2 protein. These results imply that triterpenoids possess potential antitumor activities and exert their cytotoxicity through apoptosis and G2/M cell cycle arrest. Nimbolide was found to inhibit IκB degradation and prevent nuclear translocation of NF-κB. This subsequently caused cell cycle arrest by downregulating numerous genes involved in cellular proliferation. Nimbolide can induce apoptosis through inactivation of NF-κB. This led to significant suppression of Bcl-2 with concomitant increase in the expression of Bax, cytochrome C, and Smac/DIABLO (Kavitha 2012).

The results described herein, however, are the first to demonstrate the potential of the SCNE in inhibiting the growth of CRC cells and in a xenograft nude mouse model. It was found that SCNE can mediate antitumor activity in vivo by modulating the expression of numerous tumorigenesis-related proteins. First, SCNE down-regulated the expression of Bcl-2 which is known to promote tumor survival. Second, SCNE down-regulated the expression of cyclin D1 which is known to be overexpressed in CRC and to promote tumor growth. Third, SCNE down-regulated the expression of proteins involved in tumor invasion, metastasis, and angiogenesis such as MMP-9 and MMP2. Fourth, constitutively active NF-κB and STAT3 known to regulate the expression of all of these proteins, was also inhibited by the SCNE treatment.

### REFERENCES

Arsene D, Galais M P, Bouhier-Leporrier K, Reimund J M. Recent developments in colorectal cancer treatment by monoclonal antibodies. Expert Opin Biol Ther. 2006; 6:1175-92.

Babykutty S, Priya P S, Nandini R J, Kumar M A S, Nair M S, Srinivas P and Gopala S. Nimbolide retards tumor cell migration, invasion, and angiogenesis by downregulating MMP-2/9 expression via inhibiting ERK1/2 and reducing DNA-binding activity of NF-kappa B in colon cancer cells. Molecular Carcinogenesis. 2012; 51(6):475-490.

Deeb, D.; Gao, X.; Jiang, H.; Dulchaysky, S. A.; Gautam, S. C. Oleanane triterpenoid CDDO-Me inhibits growth and induces apoptosis in prostate cancer cells by independently targeting pro-survival Akt and mTOR. Prostate 2009,69,851-860.

Fakih M. Anti-EGFR monoclonal antibodies in metastatic colorectal cancer: time for an

[symbol]

Greten F R, Eckmann L, Greten T F, Park J M, Li Z W, Egan L J, et al. IKKbeta links inflammation and tumorigenesis in a mouse model of colitis-associated cancer. Cell. 2004; 118:285-96.

Gupta S C, Prasad S, Reuter S, Kannappan R, Yadav V R, Ravindran J, et al. Modification of cysteine 179 of IkappaBalpha kinase by nimbolide leads to down-regulation of NF-kappaB-regulated cell survival and proliferative proteins and sensitization of tumor cells to chemotherapeutic agents. J Biol Chem. 2010; 285:35406-17.

Gupta S C, Reuter S, Phromnoi K, Park B, Hema P S, Nair M, et al. Nimbolide sensitizes human colon cancer cells to TRAIL through reactive oxygen species- and ERK-dependent up-regulation of death receptors, p53, and Bax. J Biol Chem. 2011; 286:1134-46.

Gupta Subhash C, Sandeo Prasad, Dhanya R. Sethumadhavan, Mangalam S. Nair, Yin-Yuan Mo and Bharat B. Aggarwal. Nimbolide, a Limonoid Triterpene, Inhibits Growth of Human Colorectal Cancer Xenografts by Suppressing the Proinflammatory Microenvironment. Clin Cancer Res. 2013; 19(16): 4465-4476. doi:10.1158/1078-0432.CCR-13-0080.

Jackson-Bernitsas D G, Ichikawa H, Takada Y, Myers J N, Lin X L, Darnay B G, et al. Evidence that TNF-TNFR1-TRADD-TRAF2-RIP-TAK1-IKK pathway mediates constitutive NF-kappaB activation and proliferation in human head and neck squamous cell carcinoma. Oncogene. 2007; 26:1385-97.

Han Yong-Seok, Lee Jun Hee, Lee Sang Hun. Fucoidan inhibits the migration and proliferation of HT-29 human colon cancer cells via the phosphoinositide-3 kinase/Akt/mechanistic target of rapamycin pathway. Molecular medicine reports, 12: 3446-3452, 2015. DOI: 10.3892/mmr.2015.3804.

Haridas, V.; Arntzen, C. J.; Gutterman, J. U. Avicins, a family of triterpenoid saponins from Acacia victoriae (Bentham), inhibit activation of nuclear factor-kappaB by inhibiting both its nuclear localization and ability to bind DNA. Proc. Natl. Acad. Sci. USA 2001; 98 (20):11557-62.

Fakih M. Anti-EGFR monoclonal antibodies in metastatic colorectal cancer: time for an individualized approach? Expert Rev Anticancer Ther. 2008; 8:1471-80. [PubMed: 18759698]

Karin M, Cao Y, Greten F R, Li Z W. NF-kappaB in cancer: from innocent bystander to major culprit. Nat Rev Cancer. 2002; 2:301-10.

Kavitha K, Priyadarsini R V, Anitha P, Ramalingam K, Sakthivel R, Purushothaman G, Singh A K, Karunagaran D and Nagini S. Nimbolide, a neem limonoid abrogates canonical NF-kappa B and Wnt signaling to induce caspasedependent apoptosis in human hepatocarcinoma (HepG2) cells. European Journal of Pharmacology. 2012; 681(1-3):614.

Kumar Harish G, Vidya Priyadarsini R, Vinothini G, Vidjaya Letchoumy P, Nagini S. The neem limonoids azadirachtin and nimbolide inhibit cell proliferation and induce apoptosis in an animal model of oral oncogenesis. Invest New Drugs. 2010; 28:392-401.

Mermelshtein A, Gerson A, Walfisch S, Delgado B, Shechter-Maor G, Delgado J, et al. Expression of D-type cyclins in colon cancer and in cell lines from colon carcinomas. Br J Cancer. 2005; 93:338-45.

Park W, Amin A R, Chen Z G, Shin D M (2013) New perspectives of curcumin in cancer prevention. Cancer Prey Res (Phila) 6: 387-400.

Singh P R, Arunkumar R, Sivakamasundari V, Sharmila G, Elumalai P, Suganthapriya E, Mercy A B, Senthilkumar K and Arunakaran J. Anti-proliferative and apoptosis inducing effect of nimbolide by altering molecules involved in apoptosis and IGF signalling via PI3K/Akt in prostate cancer (PC-3) cell line. Cell Biochemistry and Function. 2014; 32(3):217-228.

Tian, Z.; Yang, M.; Huang, F.; Li, K.; Si, J.; Shi, L.; Chen, S.; Xiao, P. Cytotoxicity of three cycloartane triterpenoids from Cimicifuga dahurica. Cancer Lett. 2005, 226 (1): 65-75.

Ying Liu, Wei CaoBo Zhang, Yong-qiang Liu, Zhong-yuan Wang, Yan-ping Wu, Xian-jun Yu, Xu-dong Zhang, Ping-hong Ming, Guang-biao Zhou, Laiqiang Huang. The natural compound magnolol inhibits invasion and exhibits potential in human breast cancer therapy. Scientific Reports 2013; 3:3098 |DOI: 10.1038/srep03098.

Youns M, Efferth T, Hoheisel J D (2009b) Microarray analysis of gene expression in medicinal plant research. Drug Discov Ther 3: 200-207.

Youns M, Efferth T, Reichling J, Fellenberg K, Bauer A, Hoheisel J D (2009a) Gene expression profiling identifies novel key players involved in the cytotoxic effect of Artesunate on pancreatic cancer cells. Biochem Pharmacol 78: 273-283. doi: 10.1016/j.bcp.2009.04.014

Youns M, Hoheisel J D, Efferth T (2010) Toxicogenomics for the prediction of toxicity related to herbs from traditional Chinese medicine. Planta Med 76: 2019-2025. doi: 10.1055/s-0030-1250432

Paul R, Prasad M, Sah N K. Anticancer biology of Azadirachta indica L (neem): a mini review. Cancer Biol Ther 2011; 12: 467-476.

1. A method of treating cancer in a human subject, the method comprising:
(a) identifying a human subject in need of treatment; and
(b) administering to the human subject a composition comprising a therapeutically effective amount of a supercritical CO₂ neem extract (SCNE), wherein the SCNE comprises nimbolide, nimbin and salinin.

2. The method of claim 1, wherein the composition further comprises a pharmaceutically acceptable excipient, wherein the pharmaceutically acceptable excipient is selected from the group di-calcium phosphate, distilled water, saline, aqueous glucose solution, alcohol (e.g. ethanol), surfactants, propylene glycol, tween-80 and polyethylene glycol; and oily carriers such as various animal and vegetable oils, white soft paraffin, paraffin, wax, glucose, fructose, sucrose, maltose, yellow dextrin, malt dextrin, white dextrin, aerosol, microcrystalline cellulose, calcium stearate, magnesium stearate, sorbitol, stevioside, corn syrup, lactose, citric acid, tartaric acid, malic acid, succinic acid, lactic acid, L-ascorbic acid, dl-alpha-tocopherol, glycerin, propylene glycol, glycerin fatty ester, poly glycerin fatty ester, sucrose fatty ester, sorbitan fatty ester, propylene glycol fatty ester, acacia, carrageenan, casein, gelatin, pectin, agar, vitamin B group, nicotinamide, calcium pantothenate, amino acids, aerated or fumed silica, calcium salts, pigments, flavors and preservatives.

3. (canceled)

4. (canceled)

5. The method of claim 1, wherein the SCNE is administered in a dosage ranging from 50 mg to 1000 mg/day.

6. The method of claim 5, wherein the amount of SCNE is about 50 mg to 1000 mg/day.

7. The method of claim 1, wherein the amount of the nimbolide present in the composition is at least 3 mg/g, the amount of the nimbin present in the composition is at least 130 μg/g nimbin; and the amount of the salinin is at least 200 μg/g.

8. The method of claim 1, wherein the SCNE comprises one or more liminoids.

9. The method of claim 1, wherein the composition further comprises one or more tocopherols; and sesame oil.

10. The method of claim 1, wherein the composition further comprises one or more tocopherols, wherein the one or more tocopherols are alpha-tocopheraol, gamma-tocopherol, vitamin E or Rosemarinus officinalis.

11. The method of claim 1, wherein the composition further comprises one or more tocopherols; sesame oil; and aerated or fumed silica.

12. The method of claim 1, wherein the composition is in a form comprising a capsule.

13. (canceled)

14. The method of claim 12, wherein the capsule is administered orally two or three times a day.

15. (canceled)

16. The method of claim 1, wherein the cancer is oral cancer or colon cancer.

17. A method of reducing at least one inflammatory cytokine in serum of a human subject in need thereof, the method comprising administering to the human subject a composition comprising a therapeutically effective amount of a supercritical CO₂ neem extract (SCNE), wherein the SCNE comprises nimbolide, nimbin and salinin.

18. The method of claim 17, wherein the amount of SCNE is about 50 mg to 75 mg or wherein the SCNE is administered in a dosage ranging from 50 mg to 1000 mg/day.

19. (canceled)

20. The method of claim 17, wherein the amount of the nimbolide present in the composition is at least 3 mg/g; the amount of the nimbin present in the composition is at least 130 μg/g; and the amount of the salinin is at least 200 μg/g.

21. The method of claim 17, wherein the SCNE comprises one or more liminoids.

22. (canceled)

23. The method of claim 17, wherein the composition comprises one or more tocopherols, wherein the one or more tocopherols are alpha-tocopheraol, gamma-tocopherol, vitamin E or Rosemarinus officinalis.

24. The method of claim 17, wherein the composition further comprises one or more tocopherols; sesame oil; and aerated or fumed silica.

25. The method of claim 17, wherein the composition is in a form comprising a capsule.

26. (canceled)

27. The method of claim 25, wherein the capsule is administered orally two or three times a day.

28. (canceled)

29. (canceled)

30. The method of claim 17, wherein the human subject has been diagnosed with oral cancer or colon cancer prior to the administering step.

31. The method of claim 17, wherein the at least one inflammatory cytokine is IFN-γ, IFN-β, TNF-α, IL-6 or IL-1.

32. The method of claim 31, wherein the at least one inflammatory cytokine is IL-6 or TNF-α.

33. The method claim 17, wherein the human subject's serum has increased levels of at least one inflammatory cytokine when compared to a reference sample before the administration of the composition comprising therapeutically effective amount of a supercritical CO₂ neem extract.

34. The method of claim 17, further comprising determining the level of at least one inflammatory cytokine in one or more cells of the human subject before the administration of the composition comprising a therapeutically effective amount of a supercritical CO₂ neem extract, wherein the level of at least one inflammatory cytokine is higher when compared to a reference sample.

35. A method of reducing inflammation in a human subject in need thereof, the method comprising administering to the human subject a composition comprising a therapeutically effective amount of a supercritical CO₂ neem extract (SCNE), wherein the SCNE comprises nimbolide, nimbin and salinin.

36. (canceled)

37. The method of claim 35, wherein the human subject has been diagnosed with oral cancer or colon cancer prior to the administering step.

38. The method of claim 35, wherein the inflammation is reduced by decreasing the expression of one or more of IFN-γ, IFN-β, TNF-α, IL-6, IL-1, NF-KB, STAT3, COX1 or COX2.

39. A method of treating a hyperproliferative disorder in a human subject in need thereof, the method comprising administering to the human subject a composition comprising a therapeutically effective amount of a supercritical CO₂ neem extract (SCNE), wherein the SCNE comprises nimbolide, nimbin and salinin.

40. (canceled)

41. (canceled)

42. The method of claim 39, wherein the subject has been diagnosed with a need for treatment the disorder prior to the administering step.

43. The method of claim 39, wherein the hyperproliferative disorder is cancer.

44. The method of claim 43, wherein the cancer is oral cancer or colon cancer.

45. A method of suppressing expression of NFkB and cycloxygenase in a human subject in need thereof, the method comprising administering to the human subject a composition comprising a therapeutically effective amount of a supercritical CO₂ neem extract (SCNE), wherein the SCNE comprises nimbolide, nimbin and salinin.

46. (canceled)

47. The method of claim 45, wherein the subject has been diagnosed with a need for suppressing the expression of NFkB and cyclooxygenase prior to the administering step.

48. The method of claim 45, wherein the human subject has been diagnosed with a need for treatment of a disorder of uncontrolled cellular proliferation prior to the administering step.

49. (canceled)

50. The method of claim 48, wherein the disorder of uncontrolled cellular proliferation is a cancer.

51. The method of claim 50, wherein the cancer is oral cancer.

52. A method of suppressing expression of NFkB and cyclooxygenase in at least one cell, the method comprising the step of contacting at least one cell with an effective amount of a supercritical CO₂ neem extract (SCNE), wherein the SCNE comprises nimbolide, nimbin and salinin.

53. (canceled)

54. (canceled)

55. The method of claim 52, wherein the contacting is via administration to a subject.

56. The method of claim 55, wherein the subject has been diagnosed with a need for treatment of a disorder of uncontrolled cellular proliferation prior to the administering step.

57. A method of modifying epidermal growth factor receptor (EGFR) signaling activity in a human subject, the method comprising administering to the human subject a composition comprising a therapeutically effective amount of a supercritical CO₂ neem extract (SCNE), wherein the SCNE comprises nimbolide, nimbin and salinin.

58. (canceled)

59. (canceled)

60. The method of claim 57, wherein the subject has been diagnosed with a need for modifying EFGR signaling activity

61. The method of claim 57, wherein the modifying is inhibiting.

62. The method of claim 57, wherein the human subject has been diagnosed with a need for treatment of a disorder of uncontrolled cellular proliferation prior to the administering step.

63. (canceled)

64. The method of claim 62, wherein the disorder of uncontrolled cellular proliferation is oral cancer.

65. A method of inducing apoptosis of a cell in a human subject in need thereof, the method comprising administering to the human subject a composition comprising a therapeutically effective amount of a supercritical CO₂ neem extract (SCNE), wherein the SCNE comprises nimbolide, nimbin and salinin.

66. (canceled)

67. (canceled)

68. The method of claim 65, wherein the human subject has been diagnosed with a need for treatment of a disorder of uncontrolled cellular proliferation prior to the administering step.

69. (canceled)

70. The method of claim 68, wherein the disorder of uncontrolled cellular proliferation is a cancer.

71. The method of claim 70, wherein the cancer is colon cancer.
